(19) United States
(12) Patent Application Publication
(10) Pub. No.: US 2018/0214442 A1
(43) Pub. Date: Aug. 2, 2018

(54) METHODS FOR ATTENUATING OR PREVENTING MU(μ)-OPIOID RECEPTOR MEDIATED TOLERANCE AND OPIOID-INDUCED HYPERALGESIA

(71) Applicant: The Board of Trustees of the Leland Stanford Junior University, Palo Alto, CA (US)

(72) Inventors: Gregory Scherrer, Mountain View, CA (US); Vivianne L Tawfik, Palo Alto, CA (US); Gregory Corder, San Francisco, CA (US)

(21) Appl. No.: 15/873,762

(22) Filed: Jan. 17, 2018

Related U.S. Application Data
(60) Provisional application No. 62/447,387, filed on Jan. 17, 2017.

(51) Int. Cl. A61K 31/485; A61K 31/4468; A61K 31/137; A61P 25/04; A61P 25/02; A61P 25/36

(52) U.S. Cl. CPC A61K 31/485; A61K 31/4468; A61K 31/137; A61K 2300/00; A61P 25/02; A61P 25/36; A61P 25/04

(57) ABSTRACT

Methods are provided for attenuating or preventing μ-opioid receptor mediated tolerance and opioid-induced hyperalgesia in a subject in need of acute or chronic opioid treatment for pain or in need of opioid anesthesia.

### RELATED APPLICATION

This application claims priority and other benefits from U.S. Provisional Patent Application Ser. No. 62/447,387, filed Jan. 17, 2017, entitled “Loss of Mu Opioid Receptor Signaling in Nociceptors, But Not Microglia, Abrogates Morphine Tolerance Without Disrupting Analgesia.” Its entire content is specifically incorporated herein by reference.

### STATEMENT OF GOVERNMENT SUPPORT

This invention was made with Government support under contract W81XWH-15-1-0076 awarded by the Department of Defense and under contract DA031777 awarded by the National Institutes of Health. The Government has certain rights in the invention.

### STATEMENT OF NON-GOVERNMENT SUPPORT

This invention was made in part with grants from the Rita Allen Foundation and the American Pain Society.

### TECHNICAL FIELD OF THE INVENTION

The present invention is generally directed to therapeutic methods, and in particular to methods for attenuating or preventing μ-opioid receptor mediated tolerance and opioid-induced hyperalgesia.

### BACKGROUND

μ-Opioid receptor agonists (MOR agonists) are among the most effective analgesics for managing acute severe, perioperative and chronic pain (Carroll et al., 2004; Kalso et al., 2004). With the staggering prevalence of pain (Pizzo et al., 2011), the broad use of μ-opioid receptor agonists for pain management has increased dramatically in the past decades. However, the long-term use of MOR agonists is counteracted by their adverse effects that particularly include respiratory depression, physical dependence, analgesic tolerance and opioid-induced hyperalgesia (Chu et al., 2008). Tolerance and OIH are primary drivers of diminished pain control and dose escalation (Angst & Clark, 2006; Collett, 1998), and novel therapeutic strategies that would bolster opioid analgesia while mitigating tolerance and OIH are urgently required to improve patients' safety and to lessen their risk of addiction.

While the analgesic, psychotropic and constipating effects of opium and its principal alkaloid morphine were recognized and utilized therapeutically centuries ago (Dhawan et al., 1996), no solution, not even following the synthesis of countless μ-receptor agonists and antagonists, has ever been found to separate the analgesia component from maladaptive processes that cause opioid doses to escalate. However, in times of an ongoing opioid epidemic that causes deaths from prescription opioid overdoses every day, it is critical to implement strategies that reduce the effective concentration of an opioid needed for its therapeutic use and to reduce the development of analgesic tolerance and opioid-induced hyperalgesia. The present invention addresses this need.

### SUMMARY

The present invention is based on the discovery that the combined administration of a μ-opioid receptor agonist with a peripherally acting μ-opioid receptor antagonist from treatment start on is useful in attenuating or preventing μ-opioid receptor mediated tolerance and hyperalgesia, and in reducing the μ-opioid receptor agonist dosage required for opioid anesthesia. Because the agonist and antagonist compete for the peripheral μ-opioid receptors, and a blockade of the peripheral μ-opioid receptors is needed for such attenuating or preventing efficacy, it is desirable that the antagonist is administered prior or essentially simultaneously with the agonist. An essentially simultaneous administration also encompasses an administration of the agonist followed within minutes by the antagonist.

Methods are provided for attenuating or preventing μ-opioid receptor mediated tolerance and opioid-induced hyperalgesia in a subject in need of acute or chronic opioid treatment for pain or in need of opioid anesthesia by administering a therapeutically effective amount of a composition comprising at least one μ-opioid receptor agonist and at least one peripherally acting μ-opioid receptor antagonist either concurrently, consecutively or in essentially simultaneous sequence, optionally in contemplation of their respective pharmacokinetic properties and mean residency times so that effective concentrations of the opioid agonist and opioid antagonist exist from treatment start on. Hereby, the administration of the composition attenuates or prevents symptoms generally associated with μ-opioid receptor mediated tolerance and/or opioid-induced hyperalgesia. In case of opioid anesthesia, the administration of the composition reduces the therapeutically effective amount of the μ-opioid receptor agonist that is required to achieve the desired degree of anesthesia. Since the amount of agonist needed to achieve the desired degree of anesthesia is reduced, the pain threshold, i.e. the threshold to perceive pain, has been increased.

One aspect of the invention relates to providing a composition encompassing an opioid agonist in combination with a peripherally acting μ-opioid receptor antagonist as means to attenuate or prevent μ-opioid receptor mediated tolerance in a subject in need of acute opioid treatment for pain. Another aspect of the invention relates to providing a composition encompassing an opioid agonist in combination with a peripherally acting μ-opioid receptor antagonist as means to attenuate or prevent μ-opioid receptor mediated tolerance in a subject in need of chronic opioid treatment for pain. Yet another aspect of the invention relates to providing a composition encompassing an opioid agonist in combination with a peripherally acting μ-opioid receptor antagonist as means to attenuate or prevent μ-opioid receptor induced hyperalgesia in a subject in need of acute opioid treatment for pain. A further aspect of the invention relates to providing a composition encompassing an opioid agonist in combination with a peripherally acting μ-opioid receptor antagonist as means to attenuate or prevent μ-opioid receptor induced hyperalgesia in a subject in need of chronic opioid treatment for pain.

A further aspect of the invention relates to attenuating μ-opioid receptor mediated tolerance in a subject in need of opioid anesthesia using a combination of a MOR agent of suitable anesthesic efficacy and a peripheral MOR antagonist, whereby the addition of a peripheral MOR antagonist to the MOR agonist reduces the amount of the MOR agonist needed to achieve a therapeutic effect.

In some embodiments of the invention, the peripherally acting μ-opioid receptor antagonist is administered to the subject prior to the administration of the μ-opioid receptor agonist. In other embodiments of the invention, the peripherally acting μ-opioid receptor antagonist is co-administered to the subject simultaneously or essentially simultaneously with the μ-opioid receptor agonist. In yet further embodiments of the invention, sustained release or controlled release formulations of the peripherally acting μ-opioid receptor antagonist and/or μ-opioid receptor agonist are administered, either in essentially simultaneous sequence to each other or in co-administration.

The above summary is not intended to include all features and aspects of the present invention nor does it imply that the invention must include all features and aspects discussed in this summary.

### INCORPORATION BY REFERENCE

All publications, patent applications and patents mentioned in this specification are herein incorporated by reference to the same extent as if each individual publication or patent application was specifically and individually indicated to be incorporated by reference.

### BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates that the μ-opioid receptor (MOR) is required for morphine antinociceptive tolerance and opioid-induced hyperalgesia (OIH), but is not expressed by spinal microglia. (a-b) Behavioral indices of chronic morphine side effects: (a) analgesic tolerance (F_{3, 20}=61.26, P<0.0001) and (b) OIH (F_{3, 20}=16.96, P<0.0001) in control and MOR KO mice (n=6 mice for all groups). (c, d) Densitometry analysis of anti-CD11b immunoreactivity in spinal cord dorsal horn to assess microglial activation in control and MOR KO mice (P=0.0032). Scale bar=100 μm. (e) in situ hybridization for Oprm1 mRNA in CX3CR1-eGFP mouse spinal cord. (f) Immunohisto-chemistry for MOR protein in CX3CR1-eGFP mouse spinal cord. (g) MOR-mCherry reporter mouse spinal cord immunostained for CD11b. Scale bars=50 μm for panels e-f. (h) Example Wiggle plots for mapped reads of Oprm1 (red) in whole DRG (bottom plot) and in purified spinal microglia (top plot). Reads were registered for the partially overlapping microglial gene Ipcef1 (gray) but not Oprm1 exons 1-4 for microglia (top plot). In dorsal root ganglia (DRG), reads mapped to Oprm1 exons 1 through 4 with little to no reads for Ipcef1 (bottom plot). (i) Mapped reads for several cell types from RNA-seq transcriptome profiling of acutely purified spinal microglia in uninjured mice and in mice with Chronic Constriction Injury (CCI) of the sciatic nerve after 2 and 7 days. n=2 independent sequencing experiments per group, consisting of pooled microglia from 6 mice per experiment. One-way ANOVA+Bonferroni (a, b) and Kruskal-Wallis (d). ★ P<0.05. Error bars are mean±SEM. Overlaid points are individual subject scores. FPKM=Fragments Per Kilobase of transcript per Million mapped reads.

FIG. 2 illustrates that conditional deletion of MOR from primary afferent nociceptors does not alter nociceptive behavior or reduce systemic morphine antinociception. (a) Trpv1^{Cre} mice were crossed with Oprm^{flox/flox} mice. Exons 2 and 3 of the Oprm1 gene are flanked by loxP sites (triangles) and are excised in TRPV1 neurons expressing Cre recombinase. (b) PCR on genomic DNA showing excision of the floxed DNA fragment (363 bp). (c,d) in situ hybridization for Oprm1 mRNA and (e,f) anti-MOR immunostaining in MOR cKO mice compared to littermate controls (n=3 per genotype). (g-j) MOR co-immunostaining with CGRP in spinal laminae I and II outer in (i) control and (j) MOR cKO mice. (k,l) MOR co-immunostaining with IB4 in spinal laminae I and II outer in (k) control and (1) MOR cKO mice. (m-o) Baseline nociceptive hypersensitivity or affective-motivational behavior to (m) von Frey filament mechanical stimulation of the hindpaw, (n) increasing water bath temperatures during tail immersion, or (o) increasing hotplate temperatures on the hindpaws between MOR cKOs (n=15) and controls (n=14). (p-r) Antinociception, resulting from an acute spinal morphine administration (1 μg, intrathecal) in MOR cKOs (n=6) and controls (n=9), in response to (p) von Frey mechanical stimulation (F_{1, 13}=5.132, P=0.0412), (q) 50° C. water tail immersion (F_{1, 13}=12.53, P=0.0036), and (r) 52.5° C. hotplate (F_{1, 13}=5.827, P=0.0313). (s-u) Antinociception, resulting from an acute systemic morphine administration (10 mg/kg, subcutaneous) in MOR cKO mice (n=13) compared to controls (n=17), when assessed by (s) von Frey mechanical filaments (F_{1, 28}=18.46, P=0.0002), (h) 50° C. water tail immersion (F_{1, 28}=18.78, P<0.0001), or (i) 52.5° C. hotplate (F_{1, 28}=12.95, P<0.0001). Repeated measures Two-way ANOVA+Bonferroni. ★ P<0.05. Error bars are ±SEM. Overlaid points are individual subject scores. Scale bars=50 μm, throughout. BL=baseline.

FIG. 3 illustrates that conditional deletion of MOR from TRPV1 nociceptors prevents the onset of morphine antinociceptive tolerance and OIH. (a,d,g) Daily nociceptive behavior and opioid antinociception throughout a 10 day chronic morphine schedule (10 mg/kg, subcutaneous, once daily). Nociceptive behavior (pre-morphine BL timepoints only): von Frey: F_{1, 30}=9.863, P=0.004; tail immersion: F_{1, 30}=0.7311; hotplate: F_{1, 30}=0.2581, P=0.0615. Antinociception (post-morphine+30 min timepoints only): von Frey: F_{1, 30}=4.812, P=0.0367; tail immersion: F_{1, 30}=19.74, P<0.0001; hotplate: F_{1, 30}=17.23, P=0.0004. (b,e,h) Antinociceptive tolerance: (left panels) Maximal possible effect (MPE) for morphine antinociception from the first administration (Day 1: +30 min) compared to the last administration (Day 10: +30 min) (von Frey: F_{1, 30}=7.621, P=0.0097; tail immersion: F_{1, 30}=28.27, P<0.0001; hotplate: F_{1, 30}=12.27, P=0.0015), and (right panels) the percent change for each subject. (c,f,i) OIH: (left panels) Percent change in the pre-morphine baseline nociceptive behaviors prior to the first administration (Day 1: BL) compared to the last (Day 10: BL) (von Frey: F_{1, 30}=6.13, P<0.001; tail immersion: F_{1, 30}=23.08, P<0.0001; hotplate: F_{1, 30}=5.163, P=0.0304), and (right panels) the percent change for each subject. Control, n=19; MOR cKO, n=13. BL=baseline. Student's t Test, two-tailed (right panels of b,c,e,f,h,i). Repeated Measures Two-way ANOVA+Bonferroni (a,d,g and left panels of b,c,e,f,h,i). ★ P<0.05. Error bars are mean±SEM. Overlaid points are individual animal scores. BL=baseline.

FIG. 4. Opioid-induced spinal long-term potentiation (LTP) is initiated by presynaptic MOR in nociceptors. (a,b) MOR cKO mice crossed with a mouse line expressing Channel-rhodopsin2 (ChR2-eYFP) in a Cre-dependent manner produces expression of ChR2-eYFP in Trpv1^{Cre}+DRG nociceptor cell bodies and central terminals in the spinal cord dorsal horn. Scale bars=50 m, throughout. (c,d) Blue light-evoked (473 nm, 1.0 mW/cm², 0.2 ms, 0.05 Hz) excitatory postsynaptic currents (EPSCs) recorded in laminae I and II outer spinal neurons in slices from MOR cKO and littermate controls. Numbered inset traces correspond to individual EPSCs before, during, and after wash-out of bath applied DAMGO (500 nM; 5 min duration). Scale bar=100 pA, 10 ms. (c) DAMGO-induced depression of EPSC amplitude and rebound LTP after washout (Control+LTP; n=8/15 neurons) in control slices. (d) MOR cKO spinal neurons do not show DAMGO-induced depression of light-evoked EPSCs or rebound LTP upon DAMGO washout (n=9/9 neurons). Error bars are mean±SEM.

FIG. 5 illustrates that pharmacological blockade of peripheral MOR by methylnal-trexone bromide (MNB) dose-dependently prevents the onset of morphine antinociceptive tolerance and OIH. (a) Temporal raster plot of nociception-induced sensory-reflexive and affective-motivational behaviors in an inescapable noxious environment (enclosed 52.5° C. hotplate). Each row displays the behavioral profile over 45 s for an individual mouse given either saline (n=17), morphine (n=19), or morphine+MNB (n=10), on the first treatment (Day 1) and after chronic treatment (Day 7). (b,c) Summary of reflexive paw flinches in panel a: (b) cumulative summation and AUC analysis for Day 1 and Day 7 trials (F_{2, 43}=8.749, P=0.0006), and (c) the dose-response effect of MNB on morphine antinociceptive tolerance displayed as the percent change in AUC between trial days. (d,e) Summary of all affective-motivational behaviors in panel a: (d) cumulative summation and AUC analysis for Day 1 and Day 7 trials (F_{2, 43}=24.61, P<0.0001), and (e) the dose-response effect of MNB on morphine tolerance. (f-k) Effect of MNB co-administration at multiple doses on acute morphine antinociception for (f) mechanically-induced nociceptive paw flinches (F_{1, 42}=182.9, P<0.0001), (g) noxious thermal-induced paw attending and guarding (F_{1, 39}=279.9, P<0.0001), (h,i) antinociceptive tolerance (reflexive hypersensitivity: F_{4, 42}=3.54, P=0.0141; affective-motivational: F_{4, 40}=6.115, P=0.0006), and (j,k) OIH (reflexive hypersensitivity: F_{4, 42}=6.825, P=0.0003; affective-motivational: F_{4, 40}=3.619, P=0.0131). MNB doses 0.0-1.0 mg/kg, n=10; MNB 10 mg/kg, n=7 for reflexive tests and n=5 for affective-motivational tests. Best-fit lines were generated following non-linear regression analysis based on the % MPE for each mouse. One-way ANOVA+Bonferroni (left panels of b,d,f-k). ★ P<0.05. Error bars are mean±SEM. Overlaid points are individual animal scores. PID=post-injury day.

FIG. 6 illustrates that combination therapy of morphine and MNB delivers long-lasting antinociception, without the onset of tolerance, during perioperative and chronic pain states. (a,d,g,j) Timecourse for (a,g) nociceptive hypersensitivity and (d,j) affective-motivational behaviors following an orthotrauma (tibia fracture and bone pinning), or a peripheral nerve injury (Chronic Constriction Injury, CCI), and the effect of chronic saline (n=10 for fracture, n=5 for CCI), 10 mg/kg morphine (n=10/injury model), or 10 mg/kg morphine+10 mg/kg MNB (n=10/injury model) treatments. Treatments are first given on PID 7 and re-administered once daily until PID 14, with the exception that the Saline group is administered acute morphine only on PID 7 and PID 14. (b,e,h,k) Antinociceptive tolerance: (left panels) Maximal possible effect (MPE) for morphine antinociception from the first administration (PID 7: +30 min) compared to the last administration (PID 14: +30 min) (Fracture: von Frey, F2, 21=2.05, P=0.0023; 55° C. drop, F2, 21=6.084, P=0.0082. CCI: von Frey, F2, 27=13.8, P=0.0009; Acetone drop, F2, 27=5.976, P=0.0213), and (right panels) the percent change for each subject. (c, f, i, l) OIH: (left panels) Percent change in the pre-morphine baseline nociceptive behaviors prior to the first administration (PID 7: BL) compared to just to the last (PID 14: BL) (Fracture: von Frey, F_{2, 22}=4.253, P=0.0274; 55° C. drop, F_{2, 21}=5.347, P=0.0133. CCI: von Frey, F_{2, 27}=0.05814, P=0.9436; Acetone drop, F_{2, 27}=0.4272, P=0.6567), and (right panels) the percent change for each subject. (m,n) Schematic detailing the influence of opioid-induced nociceptor maladaptive potentiation over CNS analgesic circuits to initiate tolerance and OIH. Conditional MOR deletion or MNB blockade of MOR signaling in nociceptors abrogates this potentiation, thereby maintaining opioid analgesic efficacy and reducing OIH. Two-way ANOVA+Bonferroni (for panels c,f,i,l two separate ANOVAs were run for Pre-injury vs. PID7, and PID7 vs. PID14). Student's t Test, two-tailed (right panels of b, c, e, f, h, i, k, 1). ★ P<0.05. Error bars are mean±SEM. Overlaid points are individual animal scores. PID=post-injury day.

FIG. 7 illustrates morphine dosing schedules and behavioral testing timecourse. (a) Drug dosing schedules for repeated saline or morphine administrations. On Day 1 all mice were given an acute morphine injection (10 mg/kg, subcutaneous) to establish a baseline analgesic response in the thermal tail immersion assay. Mice were then divided into treatment groups for saline, fixed-dose morphine (10 mg/kg, once daily, subcutaneous), or escalating morphine (10, 20, 30, 40, 40, 40 mg/kg, twice daily, subcutaneous). These treatments were given for 6 days (Days 2-7). On Day 8, sensory levels were assessed prior to morphine dosing for the presence of OIH, and then after morphine dosing (an acute morphine injection of 10 mg/kg, subcutaneous) to test for the presence of analgesic tolerance. (b) Untransformed, raw sensory threshold scores for same data presented in FIG. 1, panels a and b. n=6 for all groups. ★ P<0.05. Error bars are±SEM.

FIG. 8 illustrates the validation of the anti-MOR antibodies employed herein. (a) In wild-type C57Bl/6 mice, rabbit anti-MOR antibody (1:100; Abcam) labels primary afferent terminals in the spinal cord dorsal horn as well as spinal neurons in the dorsal horn. (b) In global MOR KO mice, immunostaining is lost in the afferent terminals and dorsal horn neurons. (c) In C57Bl/6 mice, guinea pig anti-MOR (1:1,000; Neuromics) produces a similar immunostaining pattern in the dorsal horn. (d) In global MOR KO mice, this MOR-immunoreactivity is lost, demonstrating its specificity. (e, g) Both the Abcam rabbit and Neuromics guinea pig anti-MOR antibodies labelled DRG neurons, including peptidergic CGRP-expressing nociceptors, consistent with the known MOR expression pattern. (f, h) Immunostaining of MOR in DRG with both antibodies is lost in global MOR KO mice, indicating staining specificity. Scale bars=50 μm.

FIG. 9 illustrates that MOR is expressed by dorsal root ganglion (DRG) nociceptors and spinal neurons in the dorsal horn. (a) Immunohistochemistry for MOR (Abcam anti-MOR) DRG shows that MOR is predominantly expressed by CGRP+nociceptors. (b, c) MOR expression is also observed in NeuN+neurons in the spinal cord dorsal horn with (b) immunohistochemistry for MOR and in (c) MOR-mCherrry reporter mice.

FIG. 10 shows further immunohistochemical evidence that MOR is not expressed by spinal microglia, as illustrated here for CD11b+ microglia using (a) Abcam rabbit anti-MOR and (b) Neuromics guinea pig anti-MOR antibodies. Scale bars=50 am.

FIG. 11 shows mapped reads from RNA-seq transcriptome profiling of acutely purified spinal microglia from wild-type mice with complete transsection of the sciatic nerve. No contamination from other cell-types based on sub-type specific gene markers, and importantly no evidence of Oprm1 expression in microglia were observed. FPKM=Fragments Per Kilobase of transcript per Million mapped reads. Surprisingly, but in consistency with recent studies on microglia response to nerve injury, similar numbers of transcripts were observed for Aif1 (encoding Iba1) and Itgam (encoding CD11b) in mice with nerve injury and in uninjured mice, because nerve injury-induced microgliosis is predominantly the consequence of intense microglia proliferation, rather than strong amplification of gene expression in a stable population of resident spinal microglia (Thomas et al., 2008; Yu et al., 2011; Webster et al., 2015).

FIG. 12 shows RNA-sequencing (seq) reads of microglia after morphine treatment. (a) Example wiggle plots showing microglial RNA-seq reads mapped to the genomic locus containing Oprm1. Sequencing reads of microglia mRNA isolated from morphine- (bottom) or saline-injected (top) animals corresponded to FPKM values of 0.00 and 0.86, respectively. No reads were associated with Oprm1 exons 1-4, indicated by red numbers. The reads (black bars) were mapped either to introns of the Oprm1 gene, in particular to the region overlapping with Ipcef1 (a gene known to be expressed by microglia), or to the alternative exon 9 of the Oprm1 gene (red arrow). These plots are representative of 3 morphine replicates (all with Oprm1 FPKM values of 0.00) and 4 saline replicates (2 with FPKM values 0.00 and the other two with values of 1.22 and 0.86). (b) Example wiggle plot for the microglial marker Itgam (CD11b) expression from the morphine treated replicate in (a). Consistent reads across all exons indicates no three-prime bias in our sequencing results.

FIG. 13 illustrates Cre recombinase activity in dorsal root ganglion (DRG) and spinal cord of Trpv1^{Cre} mice. Trpv1^{Cre} mice were crossed with the Ai14 Cre-dependent tdTomato reporter mouse line that generates the expression of a red-fluorescent protein in all cells that express, or have expressed, Cre recombinase throughout development. (a-c) Lineage map of tdTomato expression in dorsal root ganglia populations, determined by co-immunohistochemistry: (a) TRPV1, (b) CGRP, and (c) NF200. (d,e) tdTomato expression in the central terminals of DRG neurons, but not in spinal cord neurons.

FIG. 14 illustrates MOR expression in brain of cKO mice. MOR immunoreactivity is similar between (a) control and (b) MOR cKO mice in pain-related brain regions. Scale bar=50 μm.

FIG. 15 illustrates spinal neurons without opioid-induced long-term potentiation (LTP). (a) Representative spinal cord neurons from control mice showing DAMGO-induced depression of blue light-evoked (473 nm, 1.0 mW/cm², 0.2 ms, 0.05 Hz) EPSCs, but without DAMGO-washout rebound LTP. Numbered inset traces correspond to individual EPSCs before, during, and after wash-out of bath applied DAMGO (500 nM; 5 min duration). (b) Summary of 7 out of 15 recorded spinal neurons that did not show opioid-induced LTP. (c) Illustrative map of recorded neurons throughout the spinal cord dorsal horn for control and MOR cKO mice. Red cells are neurons that showed a rebound LTP. Gray cells are neurons that did not show a rebound LTP.

FIG. 16 illustrates murine behavioral responses in an inescapable thermal environment. (a, b) Nociceptive reflexes and (c, d) affective-motivational behaviors on a temperature controlled floor plate set at an innocuous 25° C. or a noxious 52.5° C. Behaviors were recorded during a 45 second exposure trial.

FIG. 17 illustrates chronic morphine antinociceptive tolerance and the combinatorial dose-response effect of methyl naltrexone bromide (MNB). (a) Raster plots depicting multiple behaviors displayed during the 45 second hot plate test for all dose groups. Raster data for saline, morphine+0.0 mg/kg MNB, and morphine+10.0 mg/kg MNB is the same presented in FIG. 4, panel a. (b) Visual depiction of reflexive paw flinching behavior. (c) Latencies to the first paw flinch. (d) Number of paw flinches (see FIG. 17). (e) Visual depiction of hindpaw guarding behavior. (f) Latency to the first onset of hindpaw guarding. (g) Total duration of time spent engaged in hindpaw guarding. (h) Visual depiction of hindpaw attending. (i) Latency to the first onset of hindpaw attending. (j) Total duration of time spent engaging in hindpaw attending. (k) Visual depiction of escape jumping behavior. (1) Latency to the first jump. For mice that did not jump, 45 s was used as the cutoff value. (m) Number of escape jumps. Control saline, n=17; 10 mg/kg morphine+0.0 mg/kg MNB, n=19; morphine+0.1 mg/kg MNB, n=10; morphine+0.5 mg/kg MNB, n=10; morphine+1.0 mg/kg MNB, n=10; morphine+10.0 mg/kg MNB, n=10. ★ P<0.05. Error bars are±SEM. Note: These are the same mice presented in FIG. 5, panels a-e.

FIG. 18 illustrates that a 10-day pharmacological blockade of peripheral MOR by methylnaltrexone bromide (MNB) prevents the onset of morphine antinociceptive tolerance and OIH. (a, b) Mechanical von Frey filament induced reflex thresholds (a) before (F_{2, 17}=31.35, P<0.0001) and (b) after (F_{2, 17}=20.01, P<0.0001) treatment. (c, d) Thermal 52.5° C. hotplate-induced affective-motivational thresholds (c) before (F_{2, 17}=13.38, P=0.0003) and (d) after (F_{2, 17}=24.34, P<0.0001) treatment. For all panels, Morphine, n=6; MNB 1 mg/kg+morphine, n=7; MNB 10 mg/kg+morphine, n=7. Repeated Measures 2-way ANOVA+Bonferonni. ★ P<0.05. Error bars are mean±SEM.

FIG. 19 illustrates that morphine-MNB combination treatment prevents the development of antinociceptive tolerance and OIH for mechanically-induced affective-motivational behaviors in tibia fracture and bone pinning model of orthotrauma chronic pain. (a) Timecourse of affective-motivational behaviors (paw attending and guarding) in response to a one-second von Frey filament stimulation (0.07, 0.4, 2.0 g) following leg fracture and bone pinning, and the effect of chronic saline (n=10), morphine (10 mg/kg) (n=10), or morphine+MNB (10 mg/kg) (n=10) treatments. Treatments are first given on PID 7 and re-administered once daily until PID 14, with the exception that the saline group is administered acute morphine only on PID 7 and PID 14. (b) Morphine antinociceptive tolerance and (c) OIH are significantly reduced when given in combination with MNB. Student's t Test, two-tailed (right panels of b and c). Two-way ANOVA+Bonferonni (left panels of b and c). ★ P<0.05. Error bars are mean±SEM. Overlaid points are individual animal scores. PID=post-injury day. Note: These are the same mice presented in FIG. 6, panels a-f.

### DETAILED DESCRIPTION

Methods are provided for attenuating or preventing μ-opioid receptor mediated tolerance and opioid-induced hyperalgesia in a subject in need of acute or chronic opioid treatment for pain or in need of opioid anesthesia by administering a therapeutically effective amount of a composition comprising at least one μ-opioid receptor agonist and at least one peripherally acting μ-opioid receptor antagonist either concurrently, consecutively or in essentially simultaneous sequence, optionally in contemplation of their respective pharmacokinetic properties and mean residency times.

Before describing these specific embodiments of the invention, it will be helpful to set forth definitions that are used in describing the present invention.

### I. Definitions

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “analgesia,” as used herein, refers to the feeling of pain relief from noxious stimuli, and is herein interchangeably used with the term “antinociception.”

The term “opioid anesthesia,” as used herein, refers to the use of a μ-opioid receptor agonist as an anesthetic.

The term “effective” or “therapeutically effective,” as used herein, means sufficient to achieve a desired outcome or sufficient to bring about a desired action.

The term “attenuating,” as used herein, means reducing in extent or severity.

The term “preventing,” as used herein, means precluding the occurrence of a certain event.

The term “pharmaceutically acceptable vehicle,” as used herein, refers to any excipient or carrier material that, when combined with an active ingredient such as an agonist and antagonist, allows the active ingredient(s) to retain biological activity and does not interfere with a subject's immune system.

The term “μ-opioid receptor agonist” or “agonist,” as used herein, refers to any molecule that induces μ-opioid receptor biological activity and elicits a cellular response, including downstream pathways that are induced by μ-opioid receptor signaling.

The term “μ-opioid receptor antagonist” or “antagonist,” as used herein, refers to any molecule that competitively, by competing with an agonist for the same μ-opioid receptor binding sites, or non-competitively hinders, prevents, neutralizes, reduces or abolishes biological activity of a μ-opioid receptor.

The term “peripheral antagonist,” as used herein, refers to a μ-opioid receptor antagonist that only acts on peripheral μ-opioid receptors, but not on central μ-opioid receptors.

The term “ab initio,” as used herein, means from treatment start on.

### II. Ways of Making and Using the Invention

The present invention teaches that the combined administration of a μ-opioid receptor agonist with a peripherally acting μ-opioid receptor antagonist from treatment start on is useful in attenuating or preventing μ-opioid receptor mediated tolerance and hyperalgesia, and in reducing the μ-opioid receptor agonist dosage required for opioid anesthesia. Despite the existence of μ-opioid receptor antagonists and their use in neutralizing opioid overdosing and in reversing opioid-associated constipation, finding a solution to prevent the development of μ-opioid receptor mediated tolerance and hyperalgesia that treating physicians can implement in their daily analgesia and anesthesia practice has so far been elusive. Under the current opioid crisis, a solution to address μ-opioid receptor mediated tolerance and hyperalgesia and a solution to reduce μ-opioid receptor agonist dosages, as provided herein, is not only useful, but urgently needed to save lives and to improve the quality of life of individuals who rely on opioids to get them through their day.

In order to further an understanding of the invention, a more detailed discussion is offered below regarding opioid receptors, the underlying etiology of μ-opioid receptor mediated tolerance and opioid-induced hyperalgesia, and the benefits of an ab initio combination treatment approach with a μ-opioid receptor agonist and peripherally acting μ-opioid receptor antagonist to counteract the development of tolerance and hyperalgesia via a peripheral μ-opioid receptor blockade from treatment start on.

### Opioid Receptors

Opioid agonists and antagonists have been found to interact with multiple subtypes of opioid receptors that so far have been identified and termed as k (kappa), ∂ (delta) and μ (mu) receptors (Martin, 1979; Pasternak, 2014).

Opioid analgesia results from binding to and signaling through G protein-coupled μ-opioid receptors (MORs) present along pain neural circuits (Mansour et al., 1995), and agonists of the μ-opioid receptor, such as the prototype morphine, are the most commonly used agents in pain management.

G protein-coupled receptors are widely distributed cell surface receptors responsible for signal transduction. G proteins are heterotrimeric regulatory proteins that consist of three different subunits (alpha, beta, gamma) and occur in inactive, guanosine diphosphate (GDP)-bound form or in the active, guanosine triphosphate (GTP)-bound form (Hollmann et al., 2005). The signal transduction through μ-opioid receptors involves activation and dissociation of inhibitory G_{i/o}-protein heterotrimers, inhibition of adenylyl cyclase activity, opening of K⁺ channels, and inhibition of Ca²⁺ channels (Gomez et al., 2013). Adenylyl cyclase catalyses the conversion of adenosine triphosphate (ATP) to 3,5-cyclic adenosine monophosphate (cAMP) which is instrumental in regulating numerous cell functions and modulates the activation of protein kinase A (PKA). As a consequence, short-term activation of μ-opioid receptors leads to reduced excitability and inhibition of excitatory neurotransmitter release which contributes to the opioid analgesic effect (Varga et al., 2003), however long-term activation leads to analgesic tolerance and hyperalgesia severely limiting the efficacy and safety of long-term opioid use.

### OPRM1/oprm1 Genes

The μ-opioid receptor is encoded in humans by the gene OPRM1 which is conserved in chimpanzee, Rhesus monkey, dog, cow, mouse, rat, chicken, zebrafish, and frog (National Center for Biotechnology Information, 2017).

### Nociception and Transmission of Peripheral Pain Signals Through Dorsal Root Ganglia

Nociception relates to the detection of pain-producing stimuli by afferent sensory neurons, the nociceptors, through various signalling pathways. Nociceptors are activated by threshold stimuli which are capable of causing tissue damage, and can be classified by the type of fibers that innervate the skin and that originate from cell bodies in trigeminal and dorsal root ganglia (Julius & Basbaum, 2001).

The dorsal root ganglia (DRG) are instrumental in transmitting peripheral pain signals via afferent C and A∂ nerve fibers into the dorsal horn of the spinal cord (Altier & Zamponi, 2006). μ-Opioid receptors (MORs) that are presynaptically expressed on the terminals of primary afferent neurons and on the postsynaptic neurons in the superficial dorsal horn of the spinal cord are critical for the analgesic effects of MOR agonists (Bao et al., 2015).

Action potentials traveling along these afferent fibers cause presynaptically localized calcium channels to open, thus triggering the release of neurotransmitters such as substance P, calcitonin gene-related peptide (CGRP) and glutamate (Altier & Zamponi, 2006). These neurotransmitters then activate postsynaptic receptors on neurons projecting to the higher brain centers that allow the perception of pain (Altier & Zamponi, 2006). Excessive neurotransmitter release from DRG neurons elevates postsynaptic excitation, resulting in increased pain, while inhibition of neurotransmitter release can reduce pain (antinociception), as evident from actions of endogenous opioids such as enkephalins and endorphins which activate opioid receptor signaling to inhibit the opening of (N-type) calcium channels and the consequently triggered neurotransmitter release (Altier & Zamponi, 2006).

### Opioid Analgesia and Treatment

Pain is a sensation that every person experiences at some point in life either in the form of acute pain following a traumatic injury including surgery, or in the form of repeated, chronic pain. Chronic pain is a major cause of disability and a major component of chronic illnesses ranging from cancer to arthritis, metabolic disorders and neuropathies that afflict an already substantial and ever-growing part of the population (Trang et al., 2015).

Since μ-opioid receptor agonists (opioid analgesics) are particularly effective for managing moderate to severe chronic and acute pain, they are generally the medication of choice in severe cancer-related and non-cancer related pain management. μ-Opioid receptor agonists find also frequent use in pre-, intra- and postoperative pain management for various surgeries, including but not limited to, orthognatic surgery, cesarean section, hysterectomy, gastrointestinal surgery including colorectal surgery, urology, cardiac surgery including coronary artery bypass graft surgery, tonsillectomy, prostatectomy, lumbar discectomy, vasectomy, lumpectomy, mastectomy, spinal fusion, orthopedic surgery.

Unlike local anesthetics that block any sensation, opioid analgesics selectively modulate the perception of pain without affecting basic sensations so that noxious stimuli can still be discerned but no pain can be felt (Pasternak & Pan, 2013).

### Opioid Anesthesia

Certain μ-opioid receptor agonists, including but not limited to, fentanyl, remifentanil, sufentanil, alfentanil, find use not only in pre-, intra- and postoperative pain management, but also in general and regional anesthesia practice that include cardiovascular surgeries and cesarian sections (Bovill et al., 1984). For use as an anesthetic, the agonist can be administered in various ways, including but not limited to, an intravenous bolus injection followed by continuous infusion for several minutes or hours, single intrathecal injection, intravenous single injection, often in combination with other analgesic or anesthetic agents.

### μ-Opioid Receptor Agonists

Opioid receptors can be activated by endogenous opioid peptides as well as exogenous naturally occurring or synthetically produced opioid agonists. The activation of μ-opioid receptors by endogenous or exogenous opioids results in the inhibition of N-type calcium channels which, in turn, reduces the amount of neurotransmitter released from dorsal root ganglion neurons, and mediates analgesia.

### Endogenous Opioid Peptides

Enkephalins (met- and leu-enkephalin), endorphins (ß-endorphin), and dynorphins are endogenous opioid peptides that are derived from large precursor proteins. Enkephalins are believed to primarily interact with ∂ opioid receptors (DOR), ß-endorphins with MOR, and dynorphins with k opioid receptors (KOR) (Gomez at al., 2013).

### Exogenous, Naturally Occurring Opioids

Morphine, the principal alkaloid in the opium poppy (papaver somniferum) and prototype μ-opioid receptor agonist to date, was isolated from the early 19th century on for its pain-relieving and psychotropic effects (Pasternak & Pan, 2013).

### Exogenous, Synthetically Produced Opioids

Short-acting opioids, such as fentanyl, find frequent use in outpatient and surgical procedures (Williams et al., 2013) for intraoperative pain management as well as for opioid anesthesia. Fentanyl is a powerful synthetic opioid that has found wide-spread (mis)use because of its low-cost production that provides great incentive to add it to street drugs such as heroin and counterfeit drugs such as oxycodone-containing preparations (Frank & Pollack, 2017). Other potent opioid agonists and analogs of fentanyl are alfentanil, sufentanil, remifentanil, alpha-methylfentanyl, carfentanyl, ohmefentanyl.

Long-acting opioids, such as methadone and buprenorphine, are preferably used for treating chronic pain and opioid addiction (Williams et al., 2013). Buprenorphine is also marketed together with naloxone (sublingual tablets, buprenorphine/naloxone 2 mg/0.5 mg and 8 mg/2 mg) for the maintenance treatment of opioid dependence.

Oxycodone. Oxycodone is available in immediate release oral formulations that include inactive ingredients which act as physical and chemical barriers that render it difficult to prepare injectable solutions from the oral formulation and, so, deter abuse.

Hydromorphone, also known as dihydromorphinone, and diamorphine, also known as heroine, are derivatives of morphine.

DAMGO is a synthetic enkephalin analog with high μ-opioid receptor specificity (Onogi et al., 1995).

Morphiceptin is a μ-opioid receptor selective tetrapeptide fragment that is derived from the milk protein casein and related to casomorphins, which are protein fragments derived from the digestion of casein.

### μ-Opioid Receptor Antagonists

The study of μ-opioid receptor antagonists was guided by the desire to maintain the opioid analgesic effect, but to separate away the respiratory depression and addictive properties. The opioid receptor antagonists compete with opioid receptor agonists for access to the receptors and so lessen or block the agonists' effects.

Naloxone (hydrochloride), the first opioid receptor antagonist identified, blocks all opioid receptors, but has the highest affinity for the μ-receptor. It is a long-lasting antagonist and is available in various formulations including a nasal spray version to reverse the effects of an opioid overdose. Other long-lasting antagonists are naloxazone and naloxonazine. Naltrexone, another long-lasting antagonist, is more potent, but less specific for the μ-receptor than naloxone. ß-funaltrexamine, the fumarate methy ester derivative of naltrexone, is both a μ-opioid receptor antagonist and a k-opioid receptor agonist (Dhawan et al., 1996).

Methylnaltrexone (bromide), also known as Relistor, is a cationic selective μ-opioid receptor antagonist and only peripherally active, since it cannot cross the blood-brain barrier due to its positive charge.

One of the first antagonists that was developed with the objective to reverse morphin's respiratory depression and that carried a N-allyl substitution was Nalorphine (N-allyl-normorphine).

CTOP is a representative of a group of cyclic, μ-opioid receptor selective antagonists.

Naloxegol, a polyethylene glycol (PEG) conjugated derivative of naloxone, is one of the first peripherally acting antagonists and is orally administered. Due to the PEG conjugation, naloxegol cannot cross the brain barrier to exert any central actions and its effects are restricted to the peripheral nervous system, including the enteric nervous system (Al-Huniti N et al., 2016).

Opioid-induced constipation that commonly occurs with opioid treatment has recently been treated with peripherally acting opioid antagonists including methylnaltrexone bromide and naloxegol.

### Opioid Tolerance

Opioid tolerance counteracts opioid analgesia and manifests itself when analgesic efficacy gradually decreases at fixed drug doses, requiring ever increasing doses. A typical symptom of opioid tolerance, thus, is the need for ever increasing doses to achieve the same level of analgesia. In cases of chronic opioid use, dose escalation occurs very quickly requiring higher and higher doses while the analgesic effect of the opioid diminishes.

An opioid tolerant subject is defined by the Food and Drug Administration (FDA) as a person receiving oral morphine 60 mg/day, transdermal fentanyl 25 μg/hour, oral oxycodone 30 mg/day, oral hydromorphone 8 mg/day, oral oxymorphone 25 mg/day, or an equally analgesic dose of any other opioid (Adesoye & Duncan, 2017).

### Opioid-Induced Hyperalgesia (OIH)

Typical symptoms of opioid-induced hyperalgesia are an increase in nociception (pain perception) and overall heightened sensitivity to noxious stimuli following the repeated administration of a μ-opioid receptor agonist for ambulatory pain management. An increase in acute post-operative pain following one-time high-dose intra-operative opioid use was also reported as OIH (Fletcher & Martinez, 2014).

Since OIH is also characterized by an enhanced responsiveness to noxious thermal stimulation, the transient receptor potential vanilloid type 1 (TRPV1) channel, a nonselective cation channel that is instrumental for thermal nociception induction and widely distributed in central and peripheral neurons, was suggested to play an important role in OIH (Bao et al., 2015). TRPV1 and MOR co-localize in dorsal root ganglion neurons and in the spinal cord (Bao et al., 2015).

### Cell Types and Receptors Mediating Tolerance and OIH

While opioid analgesia results from agonistic binding and signaling through μ-opioid receptors (MORs) (Matthes et al., 1996) that are present along pain neural circuits (Mansour et al., 1995), the cell types and receptors mediating tolerance and OIH remain disputed (Ferrini et al., 2013; Wang et al., 2012). Tolerance and OIH are adaptive processes proposed to result from complex alterations at the molecular level for MOR, as well as at the synaptic, cellular, and circuit levels, in both the peripheral and central nervous systems (Christie, 2008; Rivat & Ballantyne, 2016. In the adaptive process context, chronic administration of opioids modifies neuronal MOR function, including via receptor phosphorylation, signaling, multimerization, and trafficking, which may underlie tolerance and OIH (Christie, 2008; Roeckel et al., 2016). Other studies suggest that glial cells, and in particular microglia, essentially contribute to opioid tolerance and OIH (Trang et al., 2015). Chronic administration of opioids causes microglia and astrocyte activation, and interfering with glial function has been shown to reduce tolerance and OIH (Raghavendra et al., 2004; Watkins et al., 2009).

Opioids alter the properties of MOR-expressing neurons and connected nociceptive circuits at the level of the dorsal root ganglia (DRG), spinal cord dorsal horn, and brain (including in the brainstem descending pain control systems) (Christie et al., 2008; Rivat & Ballantyne 2013), but also at the level of spinal microglia, brainstem nuclei (periaqueductal grey and rostral ventromedial medulla), and sub-cortical and cortical brain regions (ventral tegmental area and anterior cingulate cortex) (Joseph et al., 2010; Zeng et al., 2006; Gardell et al., 2002; Mao & Mayer, 2001; Vera-Portocarrero et al., 2007; Cui et al., 2006; Ferrini et al., 2013; Horvath & DeLeo, 2009; Wang et al., 2009; Eidson & Murphy, 2013; Morgan et al., 2006; Vanderah et al., 2001; Connor et al., 2015; Mazei-Robison & Nestler, 2012; Ko et al., 2008).

Chronic use of μ-opioid receptor agonists recruits multiple pathways in nociceptors, many of which are also implicated in nociceptor sensitization during inflammation and transition to chronic pain (Joseph et al., 2010; Dioneia Araldi et al., 2015). Thus, while acute opioid action on MOR in nociceptors inhibits voltage-gated calcium channels (Taddese et al., 1995) and reduces the release of pronociceptive glutamate and peptides, i.e., substance P, calcitonin gene-related peptide (CGRP) (Chang et al, 1999; Kohno et al., 1999; Heinke et al., 2011; Suarez-Roca & Maixner, 1992; Miller & Hammond, 1994; Zhou et al., 2008), chronic opioid treatment and opioid withdrawal cause excitatory effects.

For example, chronic morphine administration upregulates substance P and CGRP, which could then facilitate nociception and cause OIH and tolerance (Guan et al., 2005; Tumati et al., 2011; Menard et al., 1996; Powell et al., 2003; Belanger et al., 2002; Suarezroca et al., 1992). In agreement with this idea, mice null for substance P expression showed very limited morphine tolerance (Guan et al., 2005; Scherrer et al., 2009). Antagonists for substance P receptor (NK1R) and CGRP receptor (Menard et al., 1996; Powell et al., 2003), and ablation of spinal neurons expressing NK1R (Vera-Portocarrero et al., 2007), also strongly diminish opioid tolerance.

Acute and chronic opioid use upregulates components of the adenylyl cyclase (AC)-PKA pathway (Zachariou et al., 2008; Christie, 2008), such that opioid withdrawal induces AC superactivation, which increases cAMP and PKA activities that in turn modify AMPA, NMDA, and TRPV1 receptor function (Bao et al., 2015; Chen et al., 2008; Spahn et al., 2013; Corder et al., 2013; Ruscheweyh et al., 2011).

MOR signaling in nociceptors appears to be particularly complex, as PKCs (Joseph et al., 2010; Dioneia Araldi et al., 2015), nitric oxide (Aley & Levine, 1997a; 1997b), PDGFR- (Wang et al., 2012), presynaptic NMDA receptors (Zhou et al., 2010), among many other receptor and intracellular signaling pathways, have also been shown to contribute to tolerance and OIH. Additionally excitatory MOR splice variants exist (Pasternak, 2014; Xu et al., 2014), but whether they are induced by opioids in MOR+nociceptors to cause tolerance and OIH is not clear. Specific ablation of TRPV1-expressing DRG neurons causes a profound reduction in morphine analgesic tolerance (Chen et al., 2007). In contrast, a study using MOR floxed mice found no change in tolerance following Cre expression in neurons expressing the sodium channel Nav1.8 (Weibel et al., 2013). It must be noted, however, that numerous DRG neurons, including the nociceptors that express TRPV1, but not Nav1.8 (Shields et al., 2012) still express MOR with this strategy, and that the short 4-day chronic morphine treatment might have led to incomplete tolerance.

### Methods of Attenuating or Preventing

In one aspect of the invention, methods of attenuating or preventing the development of μ-opioid receptor mediated tolerance are provided by administering to a subject, in need of acute or chronic opioid treatment for pain for in need of opioid anesthesia, compositions that comprise at least one μ-opioid receptor agonist and at least one peripherally acting μ-opioid receptor antagonist.

### Pharmaceutical Compositions for Use in the Methods of the Invention

The compositions used in the methods of the invention to attenuate or prevent μ-opioid receptor mediated tolerance and hyperalgesia comprise a therapeutically effective amount of at least one μ-opioid receptor agonist, a therapeutically effective amount of at least one μ-opioid receptor antagonist, and a pharmaceutically acceptable vehicle or excipient.

The amount of any individual excipient in the composition will vary depending on the nature and function of the excipient and particular needs of the composition. Typically the optimal amount of any individual excipient is determined through routine experimentation such as by preparing compositions containing varying amounts of the excipient, examining the stability and compatibility of constituents as well as agents, and then determining the range at which optimal performance is attained with no significant adverse effects.

Generally, the excipient(s) will be present in the composition in the amount of about 1% to about 99% by weight.

The compositions encompass all types of formulations suited for injection, infusion, nasal, dermal, subcutaneous or oral administration. They can be in the form of powders or lyophilates that can be reconstituted with a suitable diluent (water, buffered saline, saline, dextrose and similar) prior to use or already in a form that is ready for administration.

The compositions can also be provided in the form of kits with one or more containers holding compositions comprising at least one μ-opioid receptor agonist and at least one μ-opioid receptor antagonist in a liquid, ready-to-use formulation or in a dry form that requires reconstitution prior to administration with a separately packaged diluent that is also part of the kit. The kit can also comprise a package insert containing written instructions for methods of using the compositions in accordance with the present invention.

Administration of Compositions in Contemplation of their Pharmacokinetic Properties and Mean Residence Times so that Effective Blood Concentrations of Both the Opioid Agonist and the Opioid Antagonist Exist from Treatment Start on

Since the present invention is based on the combined administration of a μ-opioid receptor agonist and a peripherally acting μ-opioid receptor antagonist from treatment start on, the pharmacokinetic properties, e.g. peak blood concentrations and time-to-peak blood concentration profiles, of each combination component must be carefully considered, as because the efficacy in attenuating or preventing μ-opioid receptor mediated tolerance and opioid-induced hyperalgesia is directly related to the mean residence time (MRT) of each component in the body of a subject who receives a combined administration of a μ-opioid receptor agonist and a peripherally acting μ-opioid receptor antagonist. Optimal efficacy can be achieved, if the agonist and antagonist have closely matching MRTs in the body, meaning that their MRTs are within 50-100% percent of each other. For example, if component 1 has an MRT of 3 hours and component 2 has an MRT of 5 hours, then their MRTs are within 60% (3 hours/5 hours) of each other. Efficacy can still be achieved, albeit to a lesser degree, if the MRTs of component 1 and 2 are less closely matched, meaning that their MRTs are less than 50%.

Since many μ-opioid agonists and antagonists are highly metabolized, particularly by Cytochrome P450 (CYP) enzymes and within that group particularly by CYP3A4 enzymes, and therefore show interference with CYP3A4 inhibitors (e.g., ketoconazole) and CYP3A4 inducers (rifampicin), a change in MRTs of the combination therapy components must be taken into account if the subject, to whom the combination therapy is administered, also takes compounds with CYP3A4-inhibiting or inducing properties. CYP3A4-inhibiting properties slow down CYP-mediated metabolism and, therefore, likely increase MRT, while CYP3A4-inducing properties increase CYP-mediated metabolism and, therefore, likely decrease MRT.

Furthermore, some opioids are also metabolized by CYP2D6, and interference with CYP2D6 inhibitors and CYP2D6 inducers, respectively, must be taken into account as well.

### Formulations and Dosing Regimens for Combination Therapy of Opioid Agonist and Peripherally Acting Opioid Antagonist

In some embodiments of the present invention, the agonist and antagonist are formulated for administration by injection, e.g. intraperitoneally, intravenously, spinally, subcutaneously, intramuscularly, including auto-injector applications, etc., with pharmaceutically acceptable vehicles that are standard for injectable administration. Saline, Ringer's solution, dextrose solution, etc. are examples.

In other embodiments of the present invention, the agonist and antagonist are formulated for oral, intranasal, sublingual and buccal administration with pharmaceutically acceptable vehicles that are standard for oral, intranasal and buccal administration, and for immediate, delayed or sustained release.

### Formulations for Delayed and Sustained Release

Formulations for delayed release of orally administered agents that specifically release orally-administered agents in the small or large intestines of a subject can be made using known technology and are typically based on polymeric delivery systems.

Delayed release formulations can be formulated in tablets that can be coated using known methods to achieve delayed disintegration in the gastrointestinal tract of a subject. By way of example, a material such as glyceryl monostearate or glyceryl distearate can be used to coat tablets, or tablets can be designed for osmotically-controlled_release.

For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. Polymers capable of providing site-specific colonic delivery can also be used, wherein the polymer relies on the bacterial flora of the large bowel to provide enzymatic degradation of the polymer coat and hence release of the drug.

Delayed release formulations, as contemplated herein, delay the peak concentration of the opioid agonist and/or opioid antagonist in blood by 30 minutes to 12 hours from the time of administration of the respective agent.

Sustained release formulations are also based on polymeric systems that include, for example, polylactic-glycoloic acid (PLGA), poly-(I)-lactic-glycolic-tartaric acid (P(I)LGT), polyglycolic acid, polylactic acid, poly(ε-caprolactone) and poly(alkylene oxide). Such formulations may release an agent over a period of several hours, a day, a few days, a few weeks, or several months depending on the polymer, and the particle size of the polymer, and may be used in implants.

Sustained release formulations maintain a therapeutically effective dose of the opioid agonist and opioid antagonist for hours to days after administration of the respective agent.

The particular dosage regimen, as defined by the dosing interval and dose of agonist and antagonist, respectively, will depend on the particular formulation used for immediate, delayed or sustained release, on the particular subject, and on the subject's indication for the combination therapy (acute pain, chronic pain, opioid anesthesia, etc.). It is desirable that the most therapeutically effective doses of the combined μ-opioid agonists and antagonists (agents) are used. A therapeutically effective dose can be determined experimentally, in consideration of the agents' respective mean residence times, by repeated administration of increasing amounts of the combined agents (concomitantly or sequentially combined) in order to determine which amounts of agonist and antagonist produce a therapeutically desired endpoint.

Delayed release and sustained release formulations of agonist and antagonist in form of implantable devices are contemplated as well. Such implantable devices can comprise an agonist and an antagonist, or pharmaceutically acceptable salts or complexes thereof, and a polymer matrix, and release, upon implantation into a subject, a therapeutically effective amount of the agonist and antagonist.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### III. Experimental Procedures

The following methods and materials were used in the examples that are described further below.

### Animals

All procedures were approved by the Stanford University Administrative Panel on Laboratory Animal Care in accordance with American Veterinary Medical Association guidelines and the International Association for the Study of Pain. Mice were housed 2-5 per cage and maintained on a 12-hour light/dark cycle in a temperature-controlled environment with ad libitum access to food and water.

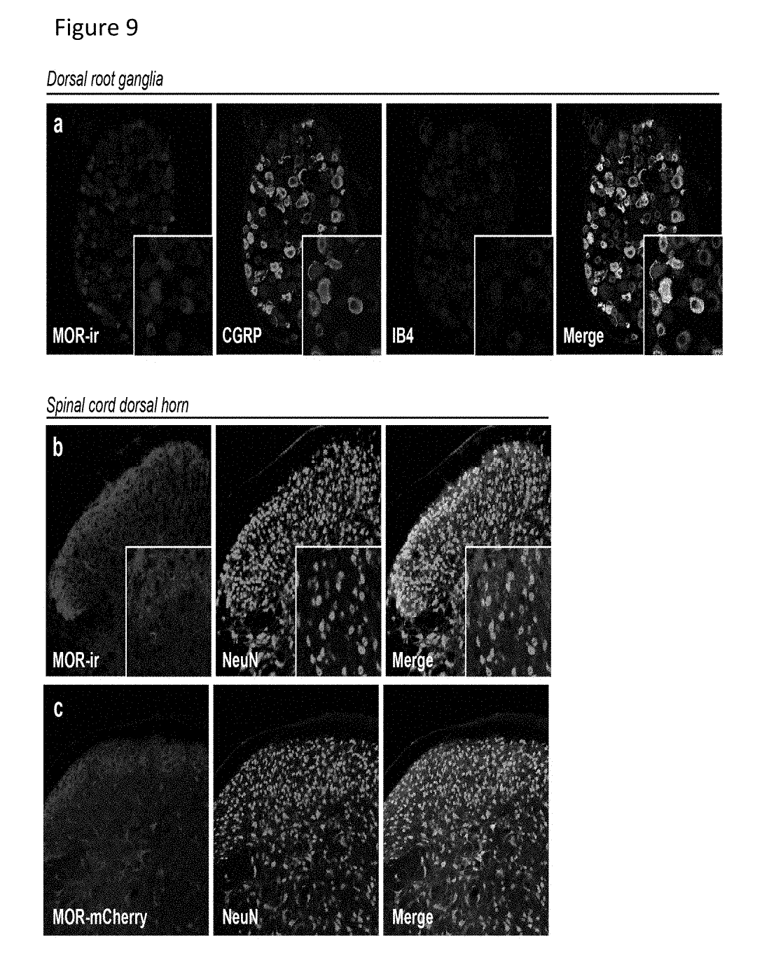

To specifically ablate the MOR in primary afferent nociceptors (MOR cKO), mice bearing a conditional allele of the Oprm1 gene containing loxP sites flanking exons 2 and 3 generated previously (Sorge et al., 2014) were crossed with transgenic mice expressing Cre under the control of the TRPV1 gene promoter, B6.129-Trpv1tm1(Cre)Bbm/J (TrpV1-cre, purchased from the Jackson Laboratory). Deletion of the Oprm1 exons 2 and 3 results in a frameshift that disrupts MOR function in MOR cKO mice. MOR cKO mice were born in the expected Mendelian ratios and showed no gross anatomical or behavioral defects, similar to the global MOR knockout mouse (Matthes et al., 1996). To confirm selective ablation of MOR in the dorsal root ganglia (DRG) only of MOR cKO mice, we performed PCR on genomic DNA from DRG, spinal cord and brain using the following primers for the excised band forward primer (5′-ACCAGTACATGGACTGGATGTGCC-3′) and reverse primer (5′-GAGACAAGGCTCTGAGGATAGTAA-3′) which resulted in a 363 bp DNA fragment that was seen in DRG but absent in spinal cord and brain (FIG. 9). Control subjects were littermate mice without the Oprm1^{flox/flox} alleles: Trpv1^{Cre}; Oprm1^{+/+}. For MOR cKO behavioral and electrophysiological studies, we used male and female mice (8-15 and 6-8 weeks, respectively). For all other experiments, we used male C57B16/J mice (8-15 weeks). Based on a power analysis, assuming 10-20% behavioral variability as in our previous work (Joseph et al., 2010), 8-12 mice per genotype is sufficient for each experimental group to reliably detect a 25% difference in means between genotypes.

### Drugs

Morphine sulfate (1 μg, intrathecal; 10-40 mg/kg/b.w., subcutaneous, West-Ward NDC 0641-6070-01, Lot#114342), methylnaltrexone bromide (0.1-10 mg/kg/b.w., subcutaneous, Salix Pharmaceuticals Inc. NDC 65649-551-07, and Sigma SML0277. Drug vehicle and dilutions used 0.9% sodium chloride (Hospira NDC 0409-4888-10, Lot#35-243-DK).

### Routes of Administration

Subcutaneous Injection.

In lightly restrained, unanesthetized mice a 30 G needle attached to a microsyringe was inserted through the skin and the drug (200-250 μl volume) is injected into the subcutaneous space.

Intrathecal Injection.

In lightly-restrained, unanesthetized mice, a 30 G needle attached to a microsyringe was inserted between the L4/L5 vertebrae, puncturing through the dura (confirmation by presence of reflexive tail flick), followed by injection of 2-5 μl as previously described (Weibel et al., 2013).

### Behavioral Testing

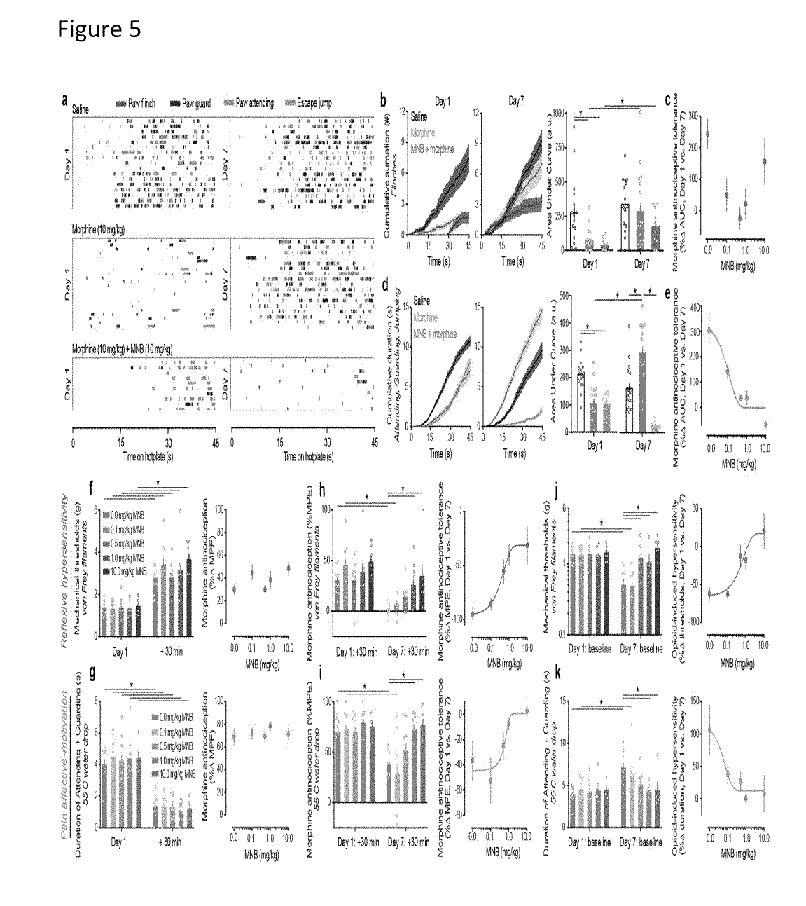

For consistency, one experimenter performed all in vivo drug administrations and behavioral testing with the exception of data in FIG. 5, panels a-e. All testing was conducted between 10:00 am-3:00 pm in an isolated, temperature- and light-controlled room. Mice were acclimated for 30-60 min in the testing environment within custom red plastic cylinders (4″ D) on a raised metal mesh platform (24″ H). The male experimenter's lab coat was present in the testing room for the first 30 minutes of acclimation, and then the experimenter entered the room for the final 30 minutes before commencement of testing to eliminate potential olfactory-induced changes in nociception (Sorge et al., 2014). The experimenter was blind to treatment and/or genotype throughout: all drugs were given to the experimenter in coded vials, and decoded only upon completion of testing. Mice of different genotypes were placed in randomized and coded holding cylinders for all sensory testing, and only after testing was the experimenter unblinded. Mice were randomized by simple selection from their homecage (5 mice per cage) prior to testing, and assigned a number. For daily testing, a secondary experimenter changed the order of the mice to be tested; the primary experimenter was blind to this order. Mice were only excluded from the study if they were found to have extensive bodily wounds from aggressions with cage mates, as the presence of additional injuries introduces potential confounds, such as alterations to the endogenous opioid system (Weibel et al., 2013).

Classification of Mouse Behaviors into Reflexive and Affective-Motivational Nociceptive Responses

To rigorously define the appearance of pain-like and pain-relief-like behaviors in non-verbal animals, a strict taxonomy was adopted, as recommended by the International Association for the Study of Pain (IASP), so as not to over-extend or inappropriately anthropomorphize rodent behaviors (Manglik et al., 2016). For example, the description of pain-like behavior in mice was limited to the use of words such as “nociceptive,” “antinociceptive,” “hypersensitivity,” “nociception-induced affective-motivational behavior,” etc., and words such as “pain,” “hyperalgesia,” “analgesia,” etc. were reserved to descriptions of the human experience and the clinical effects of opioids.

In mice, a cutaneous noxious stimulus can elicit several distinct behavioral responses (Woolf, 1984): 1. Withdrawal reflexes: rapid reflexive retraction or digit splaying of the paw that occur in response to nociceptive sensory information, but cease once the stimulus is removed and afferent nociceptive information stops; 2. Affective-motivational responses: temporally-delayed (relative to the noxious stimulus contact or removal of said stimulus), directed licking and biting of the paw (termed “attending”), extended lifting or guarding of the paw, and/or escape responses characterized by hyperlocomotion, rearing or jumping away from the noxious stimulus. Paw withdrawal reflexes are classically measured in studies of hypersensitivity, and involve spinal cord and brainstem circuits (as these behaviors are observed in decerebrate rodents only whilst the stimulus is in contact with tissue, but immediately cease once the stimulus is removed (Rescorla, 1968)). In contrast, affective-motivational responses are complex behaviors requiring processing by limbic and cortical circuits in the brain, the appearance of which indicates the subject's motivation and arousal to make the aversive sensations cease, by licking the affected tissue, protecting the tissue, or seeking an escape route (Rescorla, 1968; Blanchard & Blanchard, 1969; Bolles, 1970; Fanselow, 1982; Darwin, 1872; Chaplan et al., 1994).

### Mechanical Nociception Assays

To evaluate mechanical reflexive hypersensitivity (Weibel et al., 2013), we used a logarithmically increasing set of 8 von Frey filaments (Stoelting), ranging in gram force from 0.007 to 6.0 g. These were applied perpendicular to the plantar hindpaw with sufficient force to cause a slight bending of the filament. A positive response was characterized as a rapid withdrawal of the paw away from the stimulus filament within 4 s. Using the up-down statistical method (Solway et al., 2011), the 50% withdrawal mechanical threshold scores were calculated for each mouse and then averaged across the experimental groups.

To evaluate mechanical-induced affective-motivational responses we used three von Frey filaments (0.07 g, 0.4 g, and 2.0 g). Each filament was applied for one second to the hindpaw, and the duration of attending behavior was collected for up to 30 seconds after the stimulation. Only one stimulation per filament was applied on a given testing session, in order to prevent behavioral sensitization that can result from multiple noxious stimulations and then averaging those responses.

### Thermal Nociception Assays

Thermal Reflexive Hypersensitivity.

The tail-immersion test was used to evaluate thermal reflexive hypersensitivity (Woolf, 1984) with the temperature of the water bath being set to 48-52.5° C. The mouse was gently restrained and 2 cm of the tip of the tail was submerged in the water bath, and the latency (seconds) to reflexively withdraw the tail from the water was recorded as a positive nociceptive reflex response. A maximal cut-off of 45-60 s was set to prevent tissue damage. Only one tail immersion was applied on a given testing session, in order to prevent behavioral sensitization that can result from multiple noxious immersions and then averaging those responses.

Thermal-Induced Affective-Motivational Response.

The hotplate test was used to evaluate thermal-induced affective-motivational responses (Woolf, 1984) with the plate temperature being set to 50-52.5° C.). Mice were place on the plate and the latency (seconds) to the first appearance of an attending response (lick and/or bite at one or both hindpaws) was recorded as a positive affective-motivational response. A maximal cut-off of 45 s was set to prevent tissue damage. Only one exposure to the hotplate was applied on a given testing session, in order to prevent behavioral sensitization that can result from multiple noxious exposures and then averaging those responses.

Affective-Motivational Responses to Acute Stimulus.

To evaluate affective-motivational responses to an acute, focal thermal stimulus (Madisen et al., 2012), we applied either a single, unilateral 50 μl drop of 55° C. water or acetone (evaporative cooling) to the left hindpaw, and the duration of attending behavior was collected for up to 30 seconds after the stimulation. Only one stimulation per thermal drop was applied on a given testing session, in order to prevent behavioral sensitization that can result from multiple noxious stimulations and then averaging those responses.

Affective-Motivational Responses to Sustained Stimulus Using Modified Hot-Plate Test.

To evaluate affective-motivational responses to a sustained, inescapable noxious thermal stimulus, mice were placed on a 52.5° C. hot plate for 45 seconds. A high-speed camera (on the side, level with the hot plate floor) was used to capture the movement, speed, velocity, and detailed reflexive and affective-motivational behaviors of the mice, as described above.

As opposed to the standard hot-plate test, all behaviors such as reflexive paw flinching (rapid flicking of the limb), paw attending (directed licking of the limb), paw guarding (intentional lift protection of the limb), and escape jumping for the duration of the trial were scored. This allowed the analysis of the high-speed videos, blinded to plate temperature or treatment, for the total time spent engaging in affective-motivational behaviors and the number of nociceptive reflexives, as opposed to the traditional metric of merely determining the latency to the first reflexive response. The time the animal spent engaging in these affective-motivational behaviors reflects the degree to which the animal translates nociceptive information into an aversive signal that instructs the animal to initiate behaviors that will lessen the aversive qualities of the on-going nociceptive information (i.e. licking the tissue, and protecting the tissue by guarding or seeking escape).

Chronic Morphine Antinociceptive Tolerance Using Modified Hot-Plate Test.

To evaluate the presence of chronic morphine antinociceptive tolerance, mice were subjected to the modified hot-plate test on both the first and last day of chronic morphine dosing.

### Spinal Cord Slice Preparation and Electrophysiology

Adult mice (6-8 weeks old) were anesthetized with isoflurane, decapitated, and the vertebral column was rapidly removed and placed in oxygenated ice-cold dissection solution (in mM: 95 NaCl, 2.5 KCl, 1.25 NaH2PO4, 26 NaHCO3, 50 sucrose, 25 glucose, 6 MgCl2, 1.5 CaCl2, and 1 kynurenic acid, pH 7.4, 320 mOsm). The lumbar spinal cord was isolated, embedded in a 3% agarose block, and transverse slices (400 Lm thick) were made using a vibrating microtome (Leica V T 1200S). Slices were incubated in oxygenated recovery solution (in mM: 125 NaCl, 2.5 KCl, 1.25 NaH2PO4, 26 NaHCO3, 25 glucose, 6 MgCl2, and 1.5 CaCl2, pH 7.4, 320 mOsm) at 35° C. for 1 hour before recording. Patch-clamp recording in whole-cell configuration was performed at 32° C. on lamina I or lamina II outer zone neurons visualized with an Olympus microscope (BX51WI age-MTI). Slices were perfused at ˜2 ml/min with recording solution (in mM: 125 NaCl, 2.5 KCl, 1.25 NaH2PO4, 26 NaHCO3, 25 glucose, 1 MgCl2, 2 CaCl2, pH 7.4, 320 mOsm). Recordings were performed in voltage-clamp mode at a holding potential of −70 mV. Thick-walled borosilicate pipettes, having a resistance of 3-6 MOhm, were filled with internal solution (in mM: 120 potassium gluconate, 20 KCl, 2 MgCl2, 5 MgATP, 0.5 NaGTP, 20 HEPES, 0.5 EGTA, PH 7.25 with KOH, ˜300 mOsm). All data were acquired using a Multiclamp 700B amplifier and pClamp10 software (Molecular Devices). Sampling rate was 10 kHz and data were filtered at 2 kHz. Analysis of eEPSC peak amplitudes was done with Clampfit software (pClamp10, Molecular Devices). Graphs and statistical analysis were generated with Igor Pro (Wave Metrics).

### Optogenetic Stimulation

To isolate the TRPV1 nociceptor mediated EPSCs, we crossed the TRPV1-Cre mice with Ai32 mice (Rosa-CAG-LSL-ChR2(H134R)-EYFP-WPRE) (Bardoni et al., 2014) to limit the channelrhodopsin 2 expression specifically to TRPV1 primary afferents. The TRPV1 nociceptor mediated EPSCs were evoked by blue light (0.2 ms, 0.03 HZ) generated by an LED Transmitted Light Source (Lambda TLED, Sutter). For all recordings, 10 μM bicuculline and 2 μM strychnine were included in the recording solution to eliminate the contribution of γ-aminobutyric acid type A (GABAA) or glycine receptors on the C-fiber or TRPV1 afferent mediated EPSC.

### Immunohistochemistry

Mice (8-10 weeks) were transcardially perfused with 10% formalin in PBS. The brains, DRG (L3-L5), and spinal cord (lumbar cord L3-L5 segments) were dissected from the mice and cryoprotected in 30% sucrose in PBS. Tissues were then frozen in O.C.T. (Sakura Finetek, Inc.). Tissue sections (30 μm for brain; 40 μm for spinal cord; and 10 μm for DRG) were prepared using a cryostat (Leica Biosystems) and blocked with PBS containing 5% normal donkey serum and 0.3% Triton X-100 for 1 h at room temperature. The sections were then incubated with primary antibodies, indicated in each figure, at 4° C., overnight. For the chicken anti-GFP antibody, the incubation was performed at 37° C. for 2 h. After extensive wash with PBS containing 1% normal donkey serum and 0.3% Triton X-100, sections were incubated with appropriate secondary antibody conjugated to AlexaFluor for 2 h at room temperature. Images were collected under a Leica TCS SP5II confocal microscope with LAS AF Lite software (Leica Microsystems).

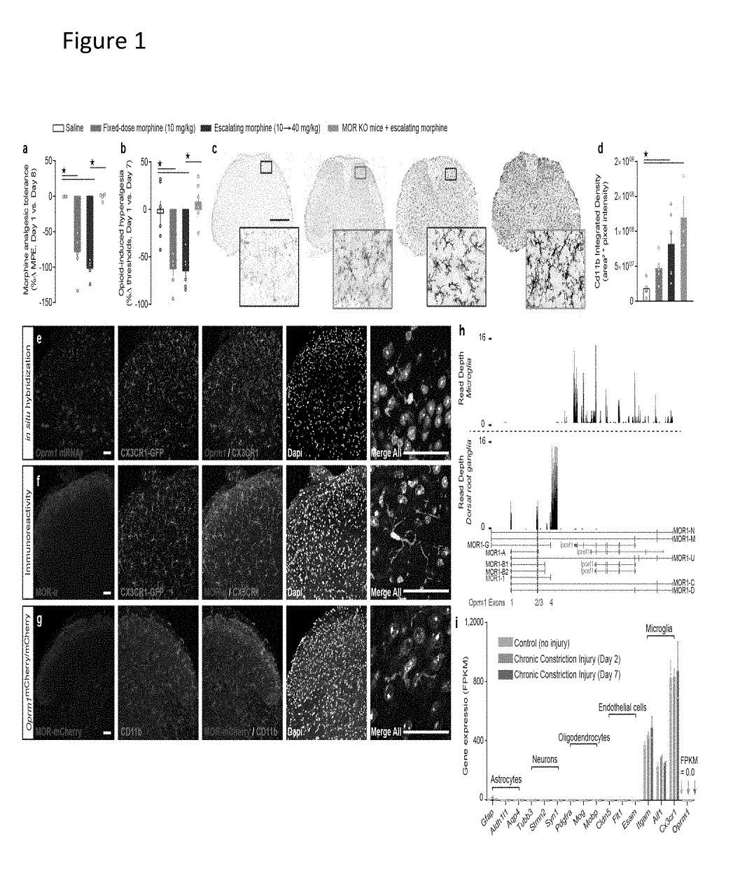

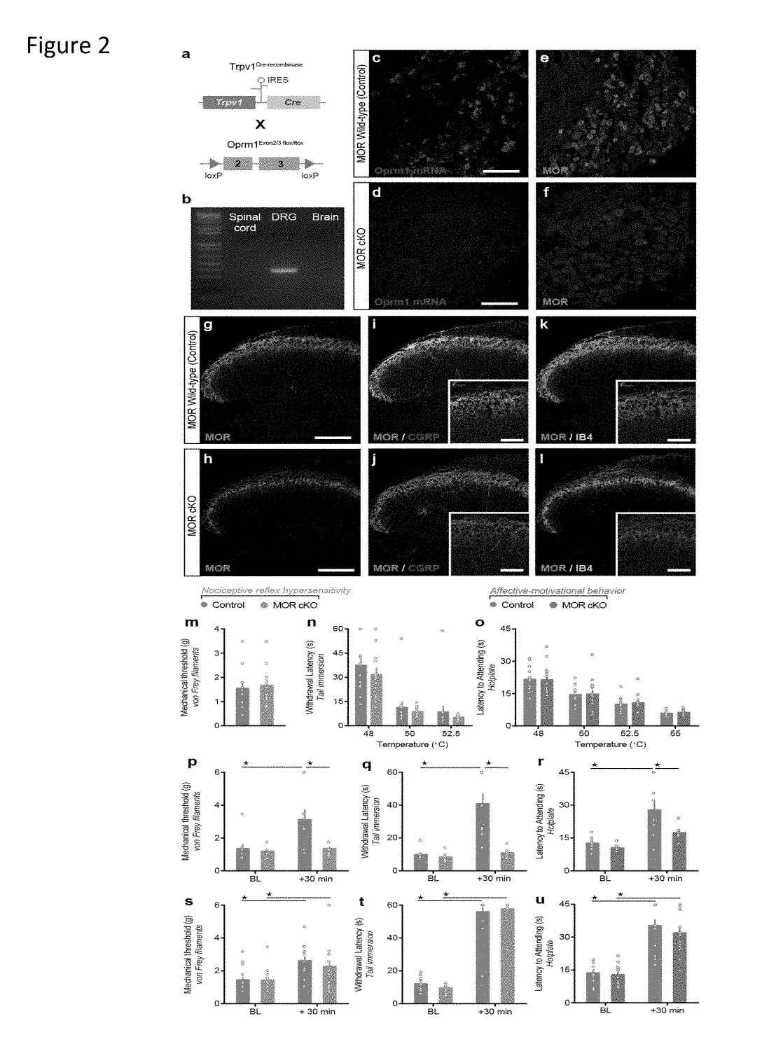

The following primary antibodies were used: anti-CD1 b, Abd Serotec # MCA711G (rat, 1:1,000), anti-CGRP: Abcam ab22560 (sheep; 1:2000); anti-GFP: Molecular Probes (rabbit; 1:1000), anti-TRPV1: gift from David Julius, UCSF (guinea-pig; 1:10000); anti-MOR used in FIG. 1: Abcam 134054 (rabbit; 1:100), anti-MOR used in FIG. 2: gift from Chris Evans, UCLA (rabbit; 1:300); anti-MOR used in FIG. 1: Abcam 10275 (rabbit; 1:100); anti-MOR: Neuromics GP10106 (guinea pig, 1:1,000). To identify IB4-binding cells, biotinylated IB4 Sigma L2140 (1:500) and fluorophore-conjugated streptavidin (Molecular Probes, 1:1000) were used in place of primary and secondary antibodies.

### In Situ Hybridization

In situ hybridization (ISH) was performed using the Panomics QuantiGene ViewRNA tissue assay (Affymetrix/Panomics), as previously described (Picelli et al., 2013). The probe set provided by Affymetrix for hybridization to the mouse mu opioid receptor (Oprm1) coding region (NM_001039652) consists of 9 blocking probes and 40 short (15-25 base pair) oligonucleotide primers upon which a branched DNA amplification “tree” is built. This methodology provides single copy sensitivity and 8,000-fold signal amplification of each target mRNA through a unique branching amplification. The signal was detected by an alkaline phosphatase reaction with the Fast Red substrate, which was visualized by bright field or fluorescent microscopy. In order to combine Oprm1 ISH with immunohistochemistry for microglial marker CD11b, the following protocol was developed: C57BL/6J wild-type mice were deeply anesthetized with isoflurane and transcardially perfused with 0.1M PBS followed by 10% formalin in PB. Lumbar DRGs were dissected, cryoprotected in 30% sucrose overnight and then frozen in OCT. Tissue was then sectioned at 12-14 μm onto Superfrost Plus slides and kept at −80° C. until use. Slides were thawed and then placed directly into 10% formalin for 10 minutes and then subsequently processed according to the manufacturer's protocol. We determined that protease treatment for 12 minutes was optimal and following this, slides were incubated for 3 hours with the RNA probe set at 40° C. After washing, pre-amplifier hybridization and hybridization with an alkaline phosphatase-based method to detect the ISH probe, the slides were blocked in 5% normal donkey serum/0.1M PBS (without Triton X-100) for one hour at room temperature and then processed for immunohistochemistry as described above.

### Microglia RNA Library Construction and Sequencing

Adult (P60) C57BL/6J mice were perfused with ice cold PBS and L4 to L6 lumbar segments of spinal cords were isolated using ribs and vertebra as landmarks. Spinal cords that retained any detectable redness (indicating presence of blood) after perfusion were discarded. Lumbar segments from 6 to 15 mice per group were pooled and mechanically dissociated in HBSS (Gibco) containing 0.5% Glucose, 15 mM HEPES pH 7.5, and 125 U/mL DNaseI (Sigma). Dissociated tissue was subjected to MACS myelin removal (Myelin Removal Beads II, Miltenyi) followed by CD11b selection (CD11b Microbeads, Miltenyi) according to the manufacturer's instructions, except all centrifugation steps were shortened to 30 s at 10,000 rcf. The isolation was performed rapidly (less than 3 hours) and care was taken to keep the cells chilled throughout to minimize gene expression changes caused by the procedure.

Total RNA was extracted from CD11b-positive cells using the RNeasy Plus Micro Kit (Qiagen). For each replicate, 10 ng of high quality total RNA (RIN>9.0) was used to prepare a poly-A enriched cDNA library using the SMARTer Ultra Low Input RNA Kit for Sequencing —v3 (Clontech). Supplemental morphine and saline treatment RNA libraries were prepared with Smart-Seq2 (Kim et al., 2015). Libraries were modified for sequencing using the Nextera XT DNA Sample Preparation Kit (Illumina) with 300 pg of cDNA as input material. Libraries were sequenced using the Illumina Nextseq to obtain 75 bp paired-end reads. Two libraries for each condition were prepared and sequenced independently for an average of 15.7 million reads per group.

Sequencing reads were mapped to the UCSC mouse reference genome mm10 essentially as previously described (Zhang et al., 2011). Mapping was accomplished by using HISAT (Quinlan & Hall, 2010) version 2.0.3 via the Galaxy platform (usegalaxy org), resulting in a minimum 70% concordant pair alignment rate. Wiggle plots were generated using BEDTools (Trapnell et al., 2010) and BedGraph-to-bigWig converter in Galaxy and the UCSC genome browser. Transcript FPKM (fragments per kilobase of transcript sequence per million mapped fragments) values were obtained using Cufflinks (Bennett & Xie, 1988) version 2.2.1 with the iGenomes mm10 reference annotation.

### Pain Models

Neuropathic Pain.

Two bilateral peripheral nerve injuries were performed: chronic constriction injury (CCI) (Bennet and Xie, 1988) and complete transsection of the sciatic nerve (Wall et al., 1979). Briefly, adult C57BL6J mice were anesthetized with isoflurane, and one sciatic nerve at a time was exposed at mid-thigh level. For CCI, two 5-0 silk sutures were loosely tied around the nerve about 2 mm apart. In the complete transsection model, a 2 mm portion of the nerve was excised. The wound was closed with tissue adhesive (Vetbond) and the procedure was repeated on the other side. Two or seven days after injury spinal cords were collected for RNAseq as described above. For behavior, injuries were performed unilaterally and mice were tested beginning seven days after the surgery.

Orthotrauma Inflammatory Pain.

Mice were anesthetized with isoflurane and underwent a distal tibia fracture and an intramedullary pin fixation in the right leg, as previously described (Wall et al., 1979). Briefly, to make the shaft for the bone pinning, a small hole was made in the proximal tibia and a 27 G needle was inserted down the medullary axis of the bone, and then removed. Next, the distal tibia was scored with a bone saw and fractured. To set the fracture, the 27 G needle was re-inserted into the intramedullary space, through the proximal tibia, and advanced across the fracture site to the distal portion of bone. The wound was closed with sterile staples. For behavior, mice were tested beginning seven days after the surgery.

### Data and Statistical Analyses

All experiments were randomized and performed by a blinded researcher. Researchers remained blinded throughout histological, biochemical, and behavioral assessments. Groups were unblinded at the end of each experiment before statistical analysis. Data are expressed as the mean±s.e.m. Data were analyzed using a Kruskal-Wallis or Student's t tests, or ordinary or repeated measures one-way or two-way ANOVA, with a Bonferroni posthoc test, as indicated in the main text or figure captions, as appropriate. For dose-response hyperalgesia studies the best-fit line was generated following non-linear regression analysis based on the % Maximum Possible Effect (MPE) for each mouse; calculated as: MPE=[(drug induced threshold−basal threshold)/basal threshold]×100.

### EXAMPLES

The following examples illustrate representative embodiments currently contemplated, but they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1: Opioid Signaling Through Mu-Opioid Receptors (MOR) on Primary Afferent Nociceptors Initiates Maladaptive Plasticity

This example illustrates that opioid signaling through MOR on neuronal (primary afferent nociceptors)—but not on glial—cells is the critical molecular event that initiates and facilitates maladaptive synaptic plasticity within nociceptive neural circuits.

Glial cells, in particular microglia, have been suggested to contribute to opioid tolerance and OIH (Trang et al., 2015). Chronic use of opioids causes microglia and astrocyte activation, and pharmacological interference with glial function was reported to attenuate opioid tolerance in rodents (Raghavendra et al., 2004; Watkins et al., 2009). Morphine has recently been proposed to act on MOR expressed by microglia to activate these cells and initiate OIH (Ferrini et al., 2013). However, unequivocal evidence for MOR expression in microglia is lacking (Kao et al., 2012), and there are conflicting reports about the involvement of microglial Toll-like receptor 4 (TLR4) and MD-2 signaling in tolerance and OIH (Watkins et al., 2009; Hutchinson et al., 2010; Mattioli et al., 2014; Fukagawa et al., 2013).

MOR function in primary afferent nociceptors is of particular interest as an initiation site for tolerance and OIH, as this cell type has been implicated in the development of antinociceptive tolerance, physical dependence, and the pronociceptive effects of opioids (Mao, 2002; Joseph et al., 2010; Chu et al., 2008. Indeed, nociceptors undergo and drive pronociceptive plasticity, in downstream CNS circuits during persistent pain (Ruscheweyh et al., 2011; Reichling & Levine, 2009). Electrophysiological studies have demonstrated that opioids not only depress neurotransmission between nociceptors and dorsal horn neurons (Heinke et al., 2011), but can also generate maladaptive plasticity, such as long-term potentiation (LTP) (Drdla et al., 2009). Opioid-induced LTP is now considered a critical neural substrate for OIH (Ruscheweyh et al., 2011), and may contribute to tolerance. The pre-versus post-synaptic origin of opioid-induced LTP is presently debated (Zhou et al., 2010; Chen et al., 2007), and it is not known whether LTP is initiated by MOR activation in nociceptors or spinal neurons. Interestingly, previous reports indicated that ablation of TRPV1 nociceptors not only abolishes opioid-induced LTP (Zhou et al., 2010), but also reduces tolerance and OIH (Chen et al., 2007).

Based on the observation that in dorsal root ganglia (DRG) MOR is predominantly expressed by peptidergic TRPV1 nociceptors (Scherrer et al., 2009; Usoskin et al., 2015), the inventors hypothesized and set out to prove that MOR expressed by nociceptors represented a critical and susceptible element within nociceptive circuits for the initiation of maladaptive mechanisms driving analgesic tolerance and OIH.

### Morphine Tolerance and OIH, but not Microglial Activation, Requires MOR

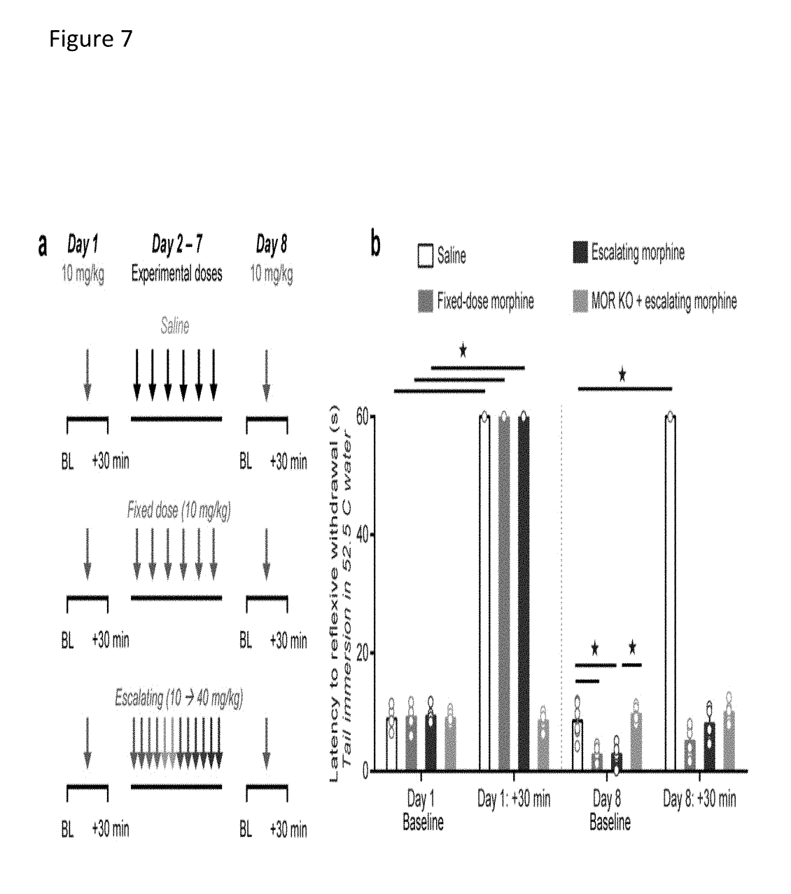

By examining, in parallel, the consequences of opioid agonist (morphine) treatment on microglial activation, analgesic tolerance, and OIH in wild-type control and global MOR KO mice, it was determined whether analgesic tolerance and OIH could be dissociated from microglial activation. In wild-type mice, with either a fixed once daily 10 mg/kg dose or with a twice daily escalating 10 to 40 mg/kg schedule, chronic morphine treatment produced significant tolerance and OIH (FIG. 1, panels a and b, and FIG. 7) as well as robust microglial activation as evidenced by increased CD11b density (FIG. 1, panels c and d). In contrast, mice that were missing the μ-opioid receptor (global MOR KO mice) and that were treated with the twice daily escalating 10 to 40 mg/kg schedule showed considerable microglial activation, but no OIH (FIG. 1, panels b and d).

Microglia Lack Oprm1 mRNA Transcript, Tagged-MOR Protein, and MOR-Immunoreactivity

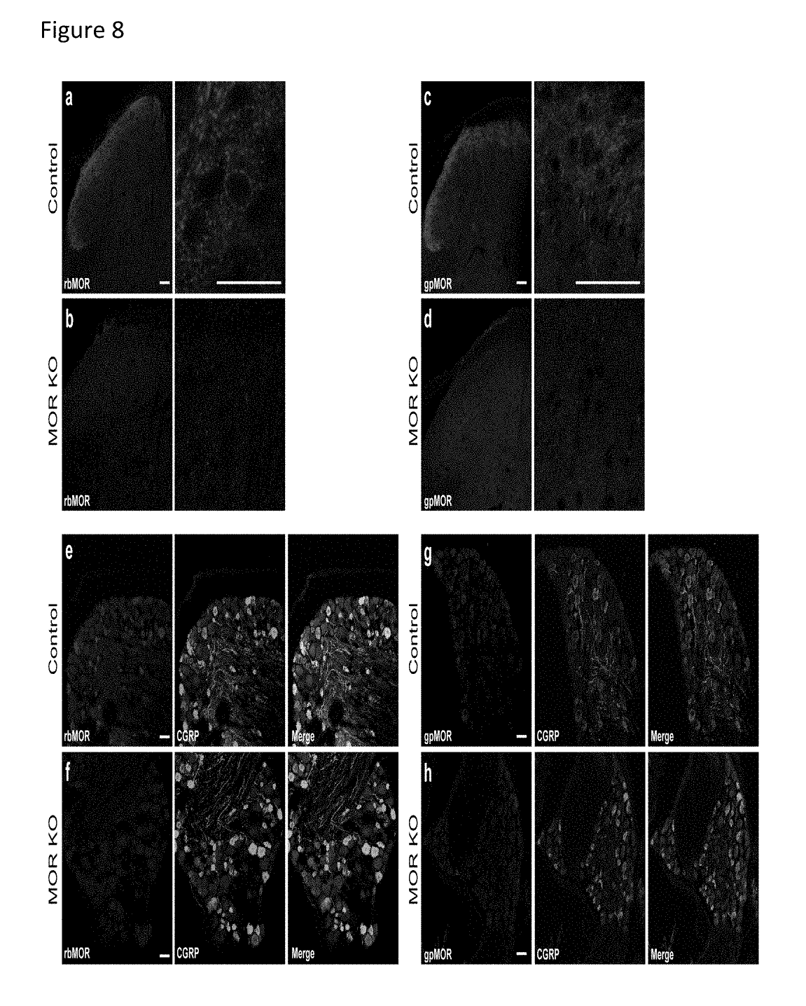

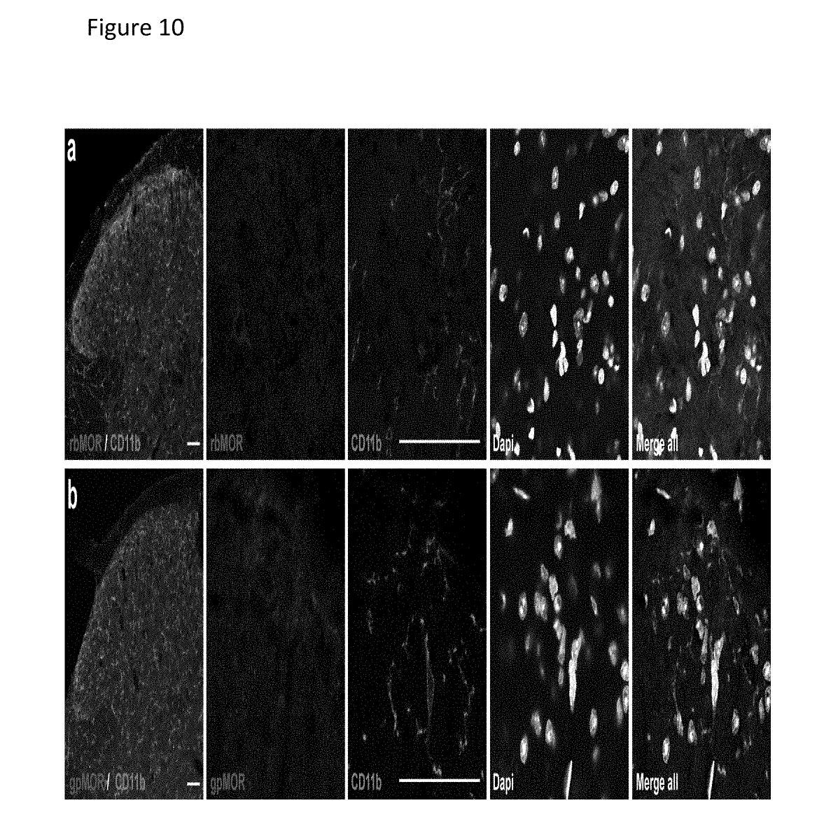

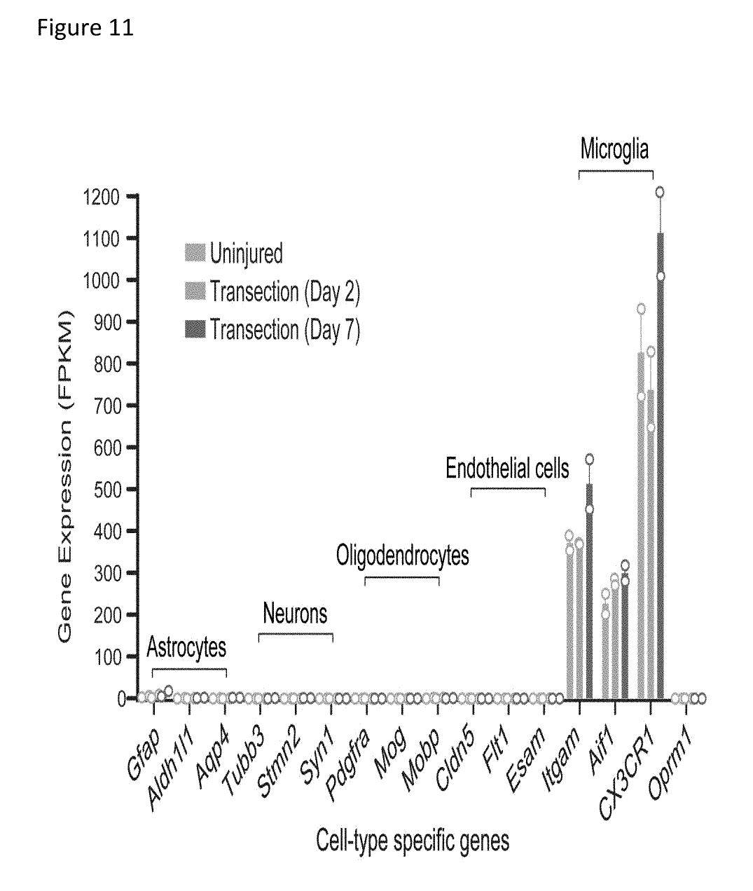

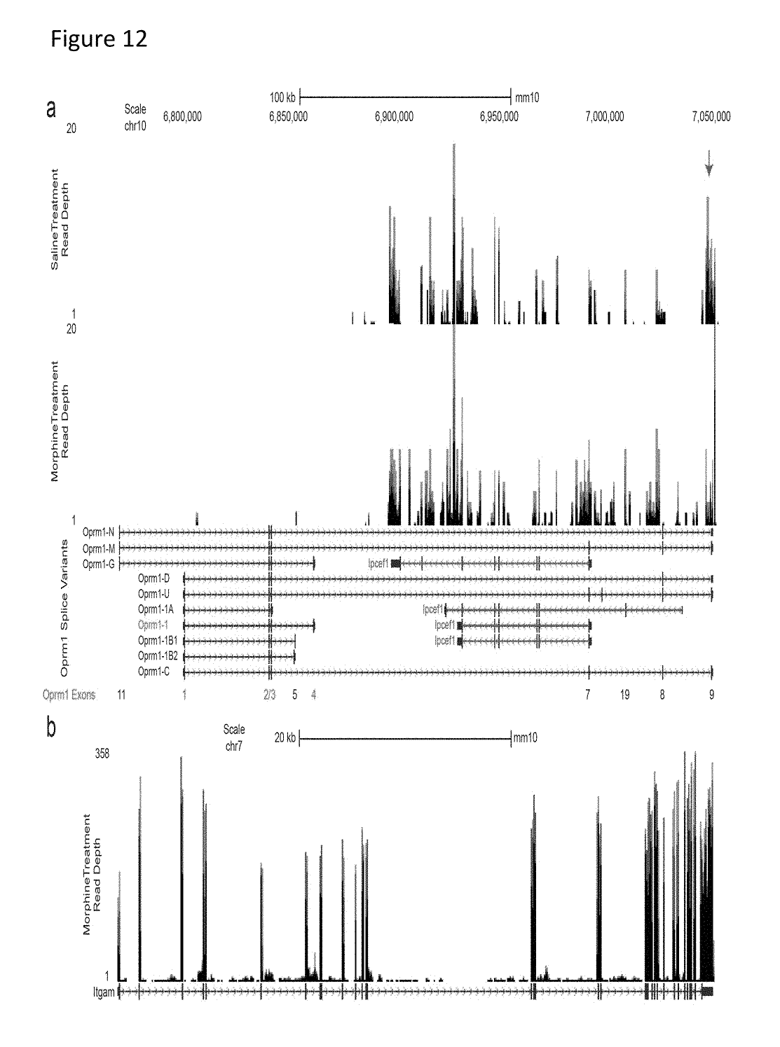

To further investigate the mechanistic separation between OIH and microglial activation and to establish which cellular populations express MOR, immunohistochemistry (IHC), in situ hybridization (ISH), and knock-in mice expressing a fluorescent tagged receptor (MOR-mCherry) were employed. MOR was found to be expressed in nociceptive neurons in dorsal root ganglion (DRG) and in spinal cord dorsal horn (FIGS. 8, 9). However, examination of dorsal horn sections provided no clear evidence for Oprm1 mRNA transcripts, anti-MOR immunoreactivity, or MOR-mCherry expression in microglia, identified by CX3CR1-GFP or CD11b immunoreactivity (FIG. 1, panels e-g, and FIG. 10). Because these results could be explained by the relatively limited sensitivity of histological and imaging techniques, RNA-sequencing (RNA-seq) transcriptome profiling was performed on acutely purified, non-cultured spinal microglia from adult mice. Hereby, expression of exons encoding the canonical seven transmembrane MOR was analyzed, as well as that of alternative splice variants and truncated receptors (Pasternak, 2014). In purified spinal microglia, unlike in DRG, there was no detection of any Oprm1 transcript (FIG. 1, panel h). RNA-seq reads were mapped within the oppositely oriented Ipcef1 gene, which partially overlaps with Oprm1, but no reads aligned to exons 1-4 of Oprm1 (which are required for the expression of the canonical seven transmembrane protein, Pasternak, 2014). There was also no detection of microglial Oprm1 transcripts mapping to exons 1-4 in mice with chronic neuropathic pain (FIG. 1, panel i, and FIG. 11), or in mice that were treated with chronic morphine (FIG. 12).

Based on the lack of microglial Oprm1 transcripts, the initiation of opioid nociceptive side effects is likely driven by MOR action in neurons.

### Example 2: Opioid Signaling Through Mu-Opioid Receptors (MOR) on Primary Afferent Nociceptors Promotes Analgesic Tolerance and Opioid-Induced Hyperalgesia

This example illustrates that chronic use of an opioid agonist induces sensitization of those MOR-expressing nociceptors promoting the development of analgesic tolerance and opioid-induced hyperalgesia.

Genetic Deletion of MOR from Nociceptors does not Reduce Systemic Morphine Antinociception

Conditional knockout mice lacking MOR in neurons of the TRPV1 lineage (MOR cKO mice) were generated in an attempt to identify the specific population of MOR+neurons that promote morphine analgesia, as well as those initiating tolerance and OIH.

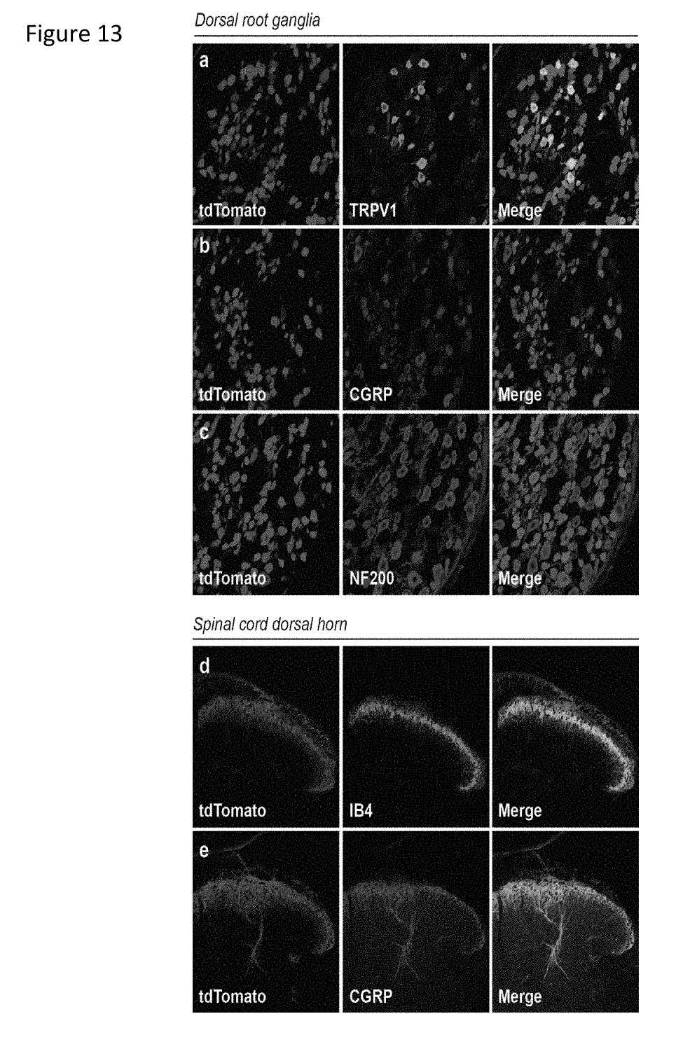

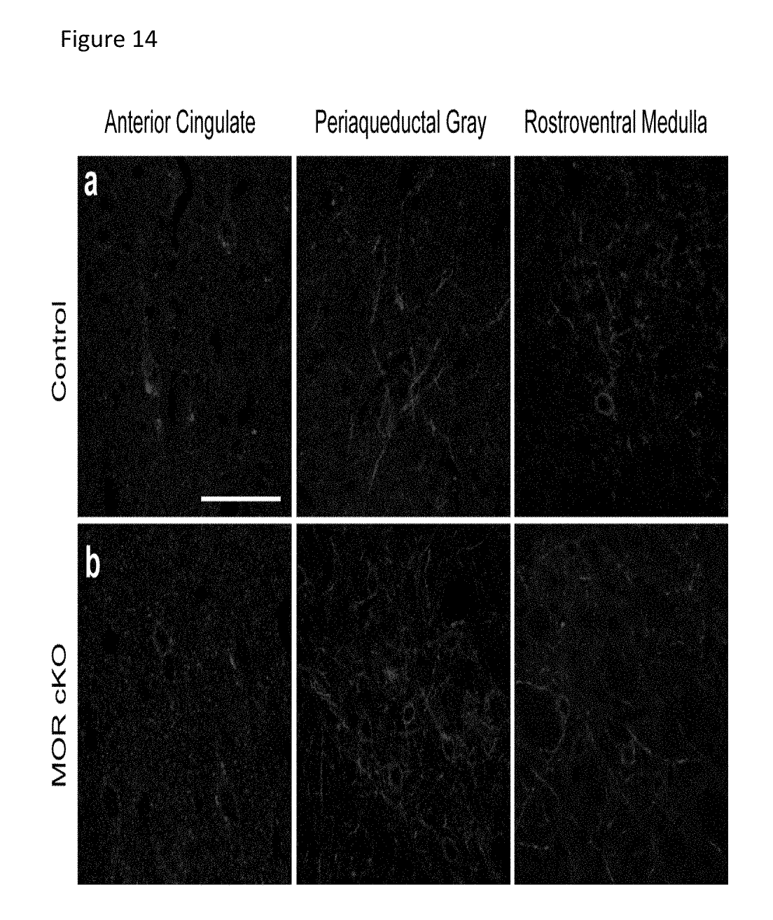

Mice bearing alleles in which exons 2 and 3 of the Oprm1 gene were floxed were crossed with knock-in mice in which expression of Cre recombinase was driven by the promoter of the Trpv1 gene (FIG. 2, panel a). TRPV1 is largely restricted to peptidergic nociceptors in adult mice, but is also expressed in non-peptidergic and myelinated nociceptors earlier during development (Cavanaugh et al., 2011), resulting in Cre-mediated recombination in most nociceptors (FIG. 13). Consequently, the Oprm1 gene was excised selectively in DRG, but not in spinal cord or brain in MOR cKO mice (FIG. 2, panel b, and FIG. 14). Fluorescence ISH and IHC experiments indicated that Oprm1 expression was detected only in 6.3% (ISH) and 4.2% (IHC) of DRG neurons in MOR cKO mice, compared to 42.6% (ISH) and 38.7% (IHC) in littermate controls (FIG. 2, panels c-f). In the spinal cord, MOR immunoreactivity was strongly decreased in the dorsal horn CGRP+ laminae I and II outer (FIG. 2, panels g-j), while a faint MOR signal persisted in the IB4+ lamina II inner (FIG. 2, panels k and 1), where MOR was expressed in spinal interneurons (Kemp et al., 1996).

Clinically, opioids provide substantive relief of both sensory and affective dimensions of the pain experience. Therefore, basal nociception and morphine antinociception were evaluated next in MOR cKO mice, monitoring both nociceptive sensory-reflexive and affective-motivational behaviors, as described in the Experimental Methods above. MOR cKO mice exhibited similar basal nociceptive reflexes and affective-motivational responses to thermal and mechanical stimuli, relative to littermate controls (FIG. 2, panels m-o). Importantly, while acute intrathecal morphine (1 μg) generated robust antinociception in control mice, intrathecal morphine did not produce significant reflexive or affective-motivational antinociception in MOR cKO mice (FIG. 2, panels p-r), revealing that spinal opioid antinociception primarily resulted from presynaptic MOR signaling in nociceptors (Heinke et al., 2011). In contrast, subcutaneous morphine (10 mg/kg) produced maximal reflexive and affective-motivational antinociception in MOR cKO mice (FIG. 2, panels s-u).

### Morphine Action at Nociceptor MORs Underlies Tolerance and OIH Initiation

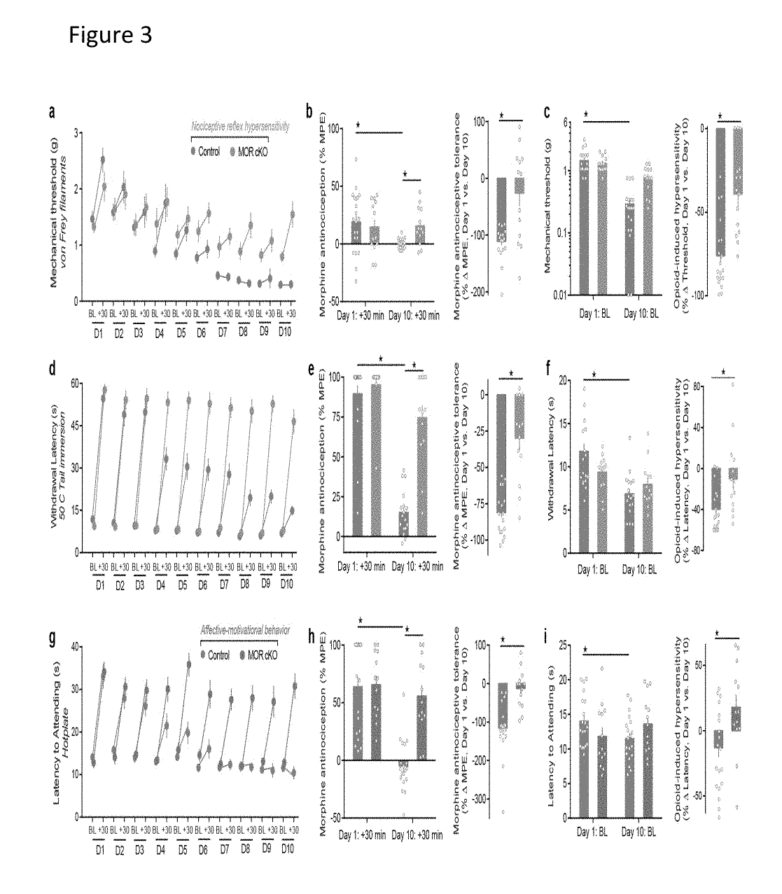

Next the development of morphine antinociceptive tolerance and OIH in MOR cKO mice was evaluated. Mice were treated with systemic, fixed-dose morphine (10 mg/kg, subcutaneous) once daily for 10 days. We measured thermal and mechanical nociceptive thresholds, and affective-motivational responses to noxious thermal stimuli prior to, and 30 min after, each daily injection, to evaluate OIH and tolerance, respectively (FIG. 3, panels a, d, g).

While morphine antinociception progressively diminished in littermate controls, morphine retained near full antinociceptive efficacy across all days in MOR cKO mice (FIG. 3, panels b, e, h). Moreover, MOR cKO mice developed significantly less OIH than controls for thermal and mechanical stimuli (FIG. 3, panels c, f, i). This result suggests that chronic opioid action at MOR expressed by nociceptors triggers the onset of pronociceptive maladaptive plasticity that results in analgesic tolerance and OIH.

### Opioids Induce a MOR-Dependent, Pre-Synaptic Form of Spinal LTP

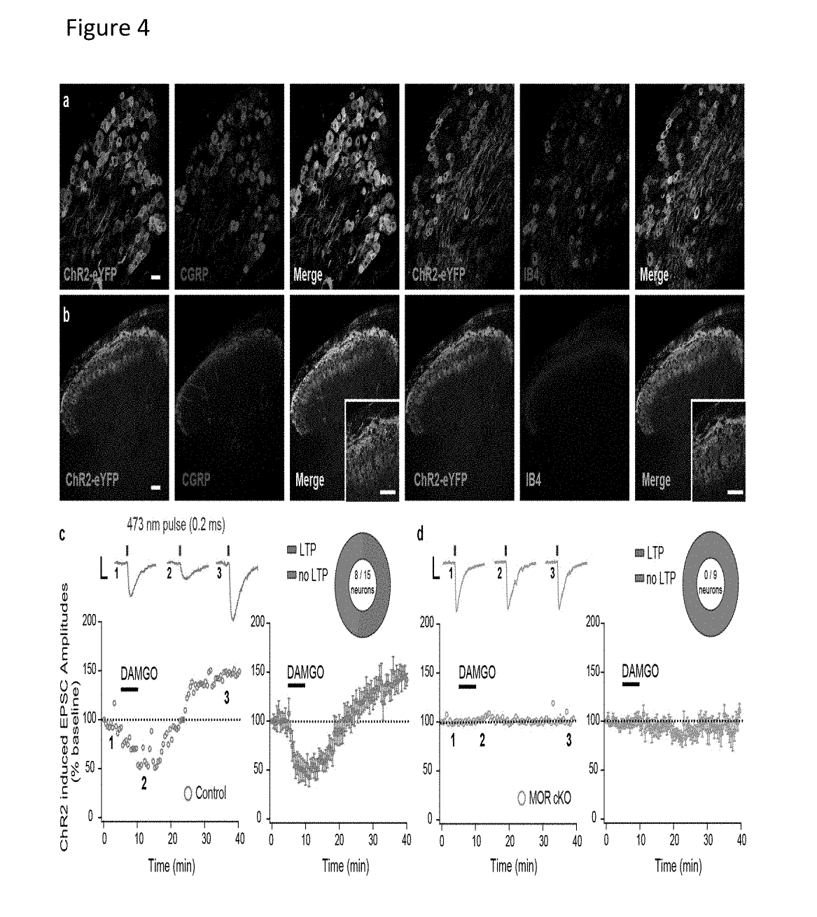

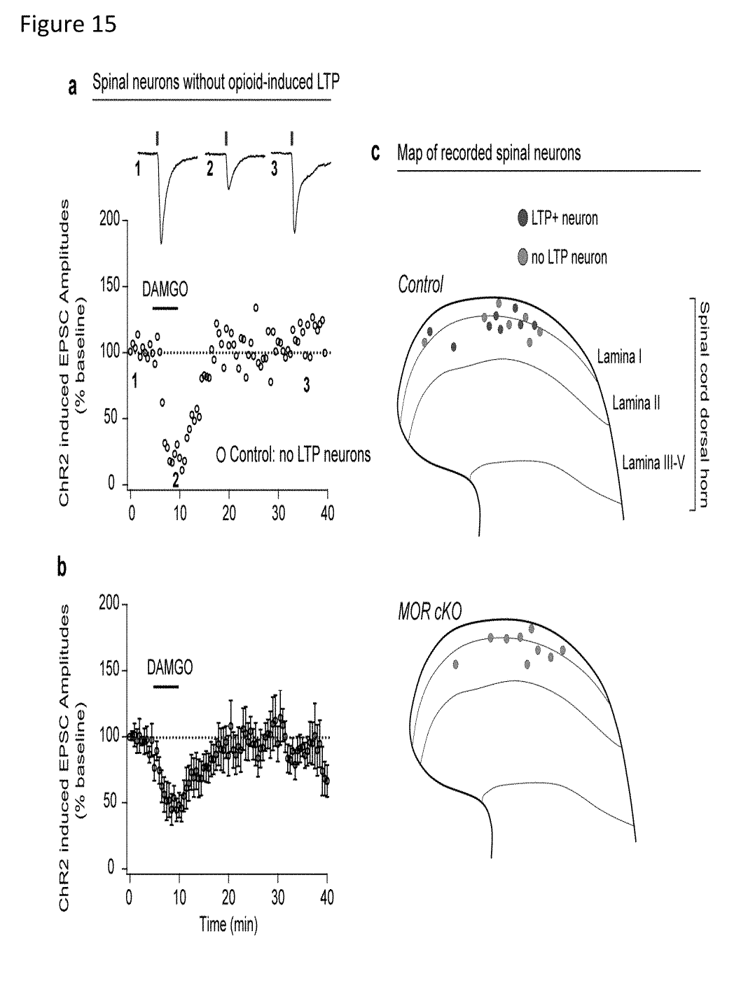

Opioids not only acutely depress synaptic transmission in the spinal cord (Heinke et al., 2011), but also trigger excitatory plasticity mechanisms such as LTP (Heinl et al., 2011; Zhao et al., 2012; Drdla et al., 2009). Because it is unclear whether opioid-induced LTP is initiated by activation of presynaptic or postsynaptic MOR signaling mechanisms, synaptic transmission between nociceptors and spinal neurons was investigated using spinal cord slices from MOR cKO mice and littermate controls expressing the light-activated channel channelrhodopsin 2 (ChR2) in TRPV1 nociceptors. Immunohistochemical studies confirmed expression of ChR2-eYFP in peptidergic DRG nociceptor somata and their terminals in lamina I and II outer (FIG. 4, panels a and b). Whole-cell recordings were obtained from lamina I-II outer neurons with monosynaptic input from ChR2-expressing nociceptors. Bath application of the MOR agonist [D-Ala², N-MePhe⁴, Gly-ol⁵]-enkephalin (DAMGO; 500 nM) for 5 min induced a rapid depression of light-evoked excitatory postsynaptic currents (EPSCs) in all 15 recorded neurons (52±10.5% inhibition at 5 min of application) (FIG. 4, panel c). Thirty minutes after washout of DAMGO, EPSC amplitudes were potentiated to 142.2±6.1% of control in 8 out of 15 neurons (FIG. 4, panel c, and FIG. 15), consistent with previous reports (Drdla et al., 2009; Zhou et al., 2010). This opioid-induced LTP persisted for the duration of the recording. Strikingly, both the DAMGO-induced inhibition and LTP were completely lost in 9 out of 9 recorded neurons from MOR cKO mice (DAMGO inhibition: 98±8.12% of control; DAMGO-washout: 96.2±11.9%) (FIG. 4, panel d). Collectively, these results establish that presynaptic MOR signaling in TRPV1 nociceptors is essential to maladaptive spinal opioid-induced LTP.

### Example 3: Disrupting/Eliminating Peripheral MOR Signaling Allows Safe and Effective Pain Control while Limiting Opioid Dose Escalation Using Opioid Agonist/Peripheral Opioid Antagonist Combinations

This example illustrates that ablation of the peripheral MOR signaling does not affect antinociception, but prevents the development of opioid tolerance and opioid-induced hyperalgesia.

### Peripheral MOR Blockade Prevents the Onset of Morphine Tolerance and OIH

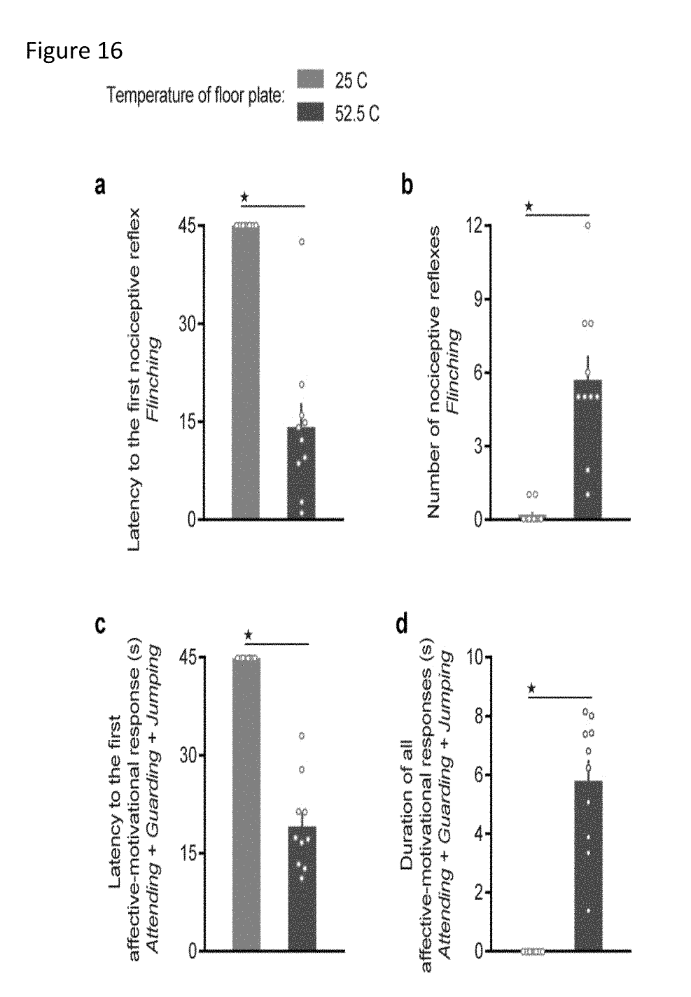

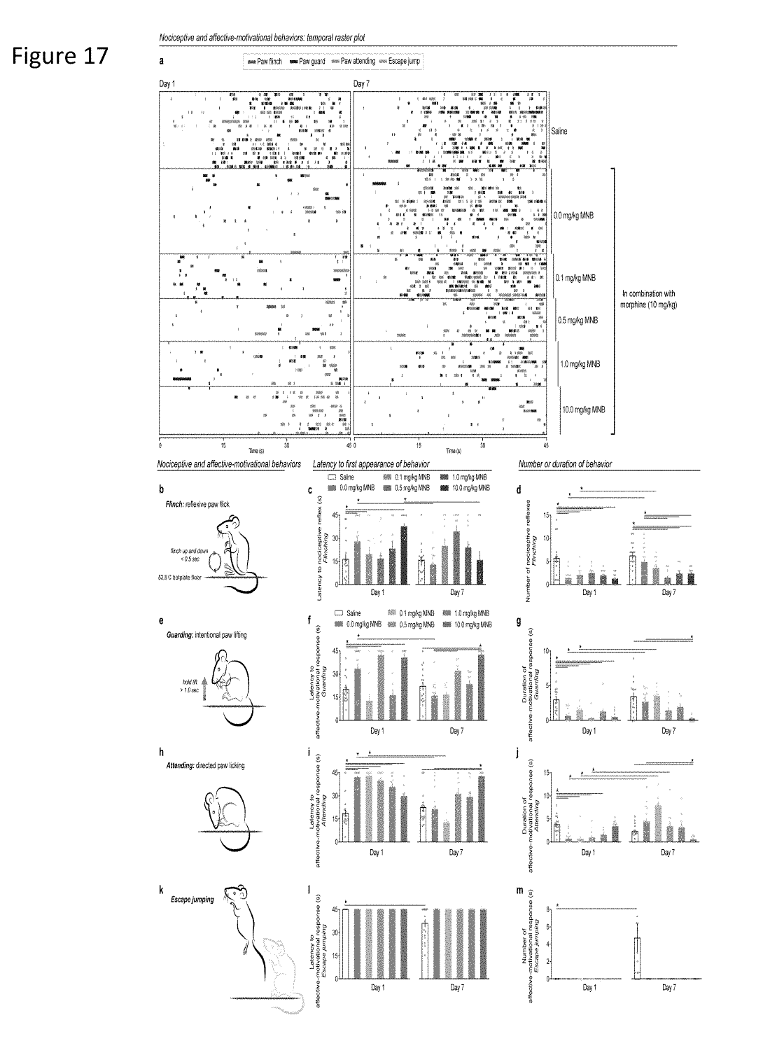

Because the described genetic Oprm1 conditional deletion strategy demonstrated that loss of MOR signaling in DRG nociceptors prevented the onset of morphine antinociceptive tolerance and OIH, blockade of peripheral MOR with a pharmacological inhibitor was investigated next. In wild-type C57Bl/6J mice, injections of subcutaneous morphine were paired with methylnaltrexone bromide (MNB), a blood-brain barrier impermeable MOR antagonist (Russell et al., 1982), and the effects of morphine and MNB combination therapy on nociceptive reflexes and affective-motivational behaviors were assessed. A paradigm was used in which the noxious environment cannot be escaped (FIG. 5, panels a-e, and FIG. 16) and pain-like behaviors elicited by acute, punctate mechanical, and noxious thermal stimuli were monitored (FIG. 5, panels f-k). Morphine was found to significantly reduce nociceptive reflexes (FIG. 5, panels a, b, c, f, and FIG. 17, panels a-d), and was also found to be effective at alleviating affective-motivational behaviors (FIG. 5, panels a, d, e, g, and FIG. 17, panels a, e-m). The combination treatment with MNB (0.1-10.0 mg/kg) did not alter morphine antinociceptive effects (FIG. 5, panels a, b, d, f, g, and FIG. 17), consistent with the results obtained in MOR cKO mice.

As shown in FIG. 17, panel c, the effect of MNB on morphine tolerance followed a bell-shaped dose-response curve only for the latency to nociceptive reflexes when scoring the number of flinches. There is clinical evidence of a similar ceiling effect of MNB on laxation responses, with a higher percentage of responders after 5 or 12.5 mg subcutaneous MNB compared to a 20 mg dose (Portenoy et al., 2008). Translation of our results therefore requires clear dose-finding studies to ensure that the dosing regimen selected is optimized to the most relevant outcomes.

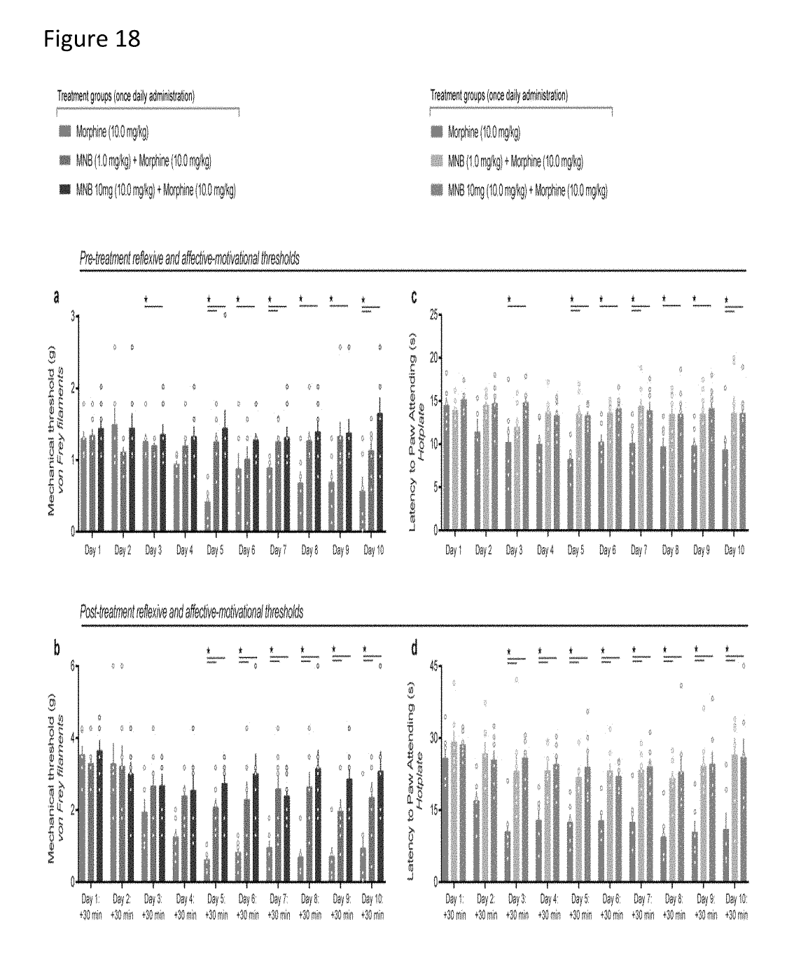

Next was determined whether MNB can effectively prevent opioid antinociceptive tolerance and OIH, by treating mice with a fixed combination of morphine (10 mg/kg) and MNB (0.1-10.0 mg/kg), or morphine alone, once daily for 7 days (FIG. 5, panels a-k). Remarkably, while mice treated with morphine alone developed robust antinociceptive tolerance and OIH on both reflexive and affective-motivational measures on Day 7 compared to Day 1 morphine antinociception and pre-morphine baseline levels, mice co-treated with morphine plus MNB showed a dose-dependent reduction in the onset of tolerance and OIH (Figure, panels 5a-k, and FIG. 18). The administration of MNB combination therapy did not produce any behavioral symptoms of physical withdrawal at any of the tested doses.

MNB and Morphine Combination Therapy Provides Long-Lasting Relief from Chronic Pain

Clinically, opioids are prescribed for managing both perioperative and chronic pain. Therefore, the antinociceptive efficacy of morphine and MNB combination therapy was investigated next in these persistent pain states using a tibia fracture and bone pinning model of orthotrauma inflammatory pain.

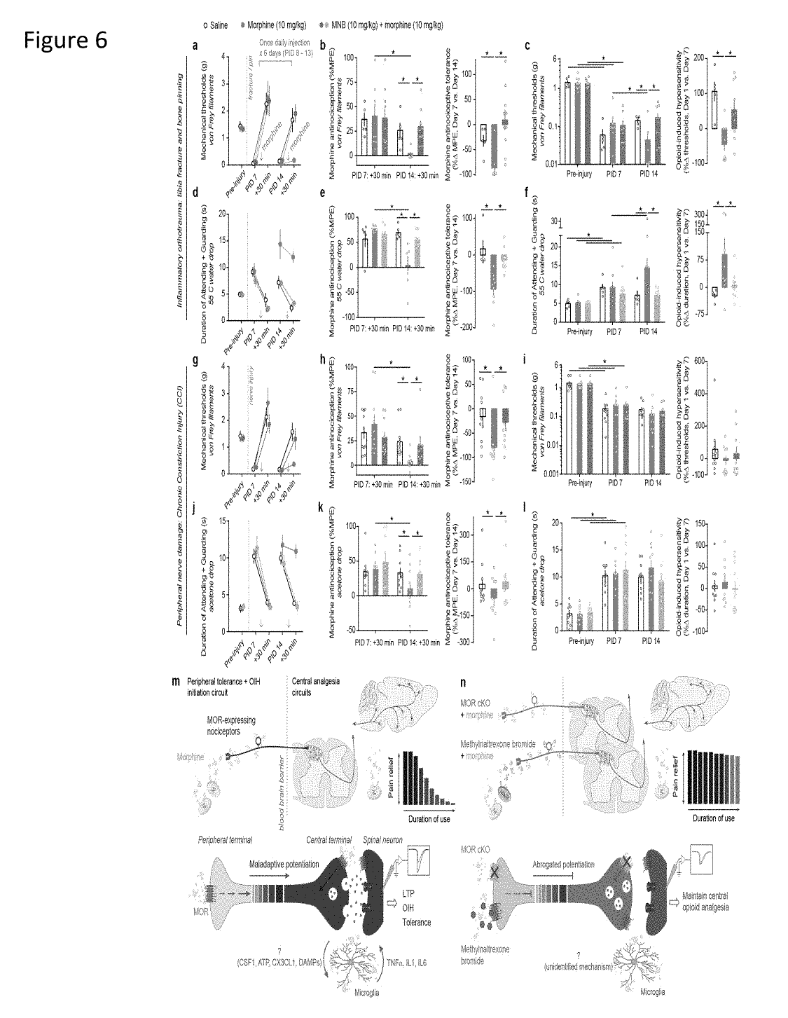

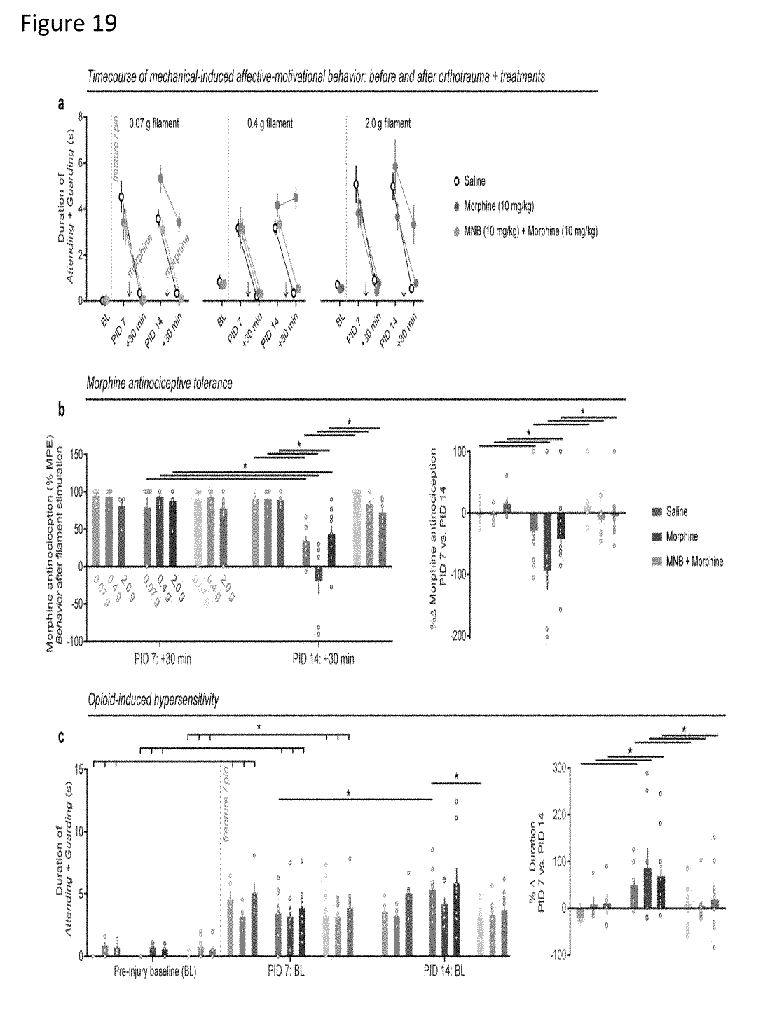

While morphine alone (10 mg/kg) acutely produced antinociception, morphine was no longer effective at reducing nociceptive or affective-motivational orthotrauma pain after 7 days of chronic treatment (FIG. 6, panels a, b, d, e). In contrast, morphine (10 mg/kg) and MNB (10 mg/kg) combination therapy produced strong antinociception against mechanical reflexive hypersensitivity, as well as against affective-motivational pain responses, with no indication of tolerance (FIG. 4, panels a, b, d, e, and FIG. 19, panels a, b). Furthermore, no OIH was observed during prolonged use of the combination therapy (FIG. 6, panels a, c, d, f, and FIG. 19, panel c). Similarly, morphine and MNB combination therapy prevented the development of antinociceptive tolerance in the chronic constriction injury (CCI) model of neuropathic chronic pain (FIG. 6, panels g-l). No indications of OIH were observed in the control group (CCI+morphine alone), possibly due to floor and/or ceiling effects (FIG. 6, panels i and l). Together, these pharmacological results uncover the potential benefit of a peripherally restricted opioid antagonist to limit detrimental pronociceptive side effects that accompany prolonged opioid use.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Adesoye A & Duncan N. Acute Pain Management in Patients With Opioid Tolerance. US Pharm. 42(3): 28-32 (2017).
Al-Huniti N, Chapel S, Xu H, Bui K H, Sostek M. Population pharmacokinetics of naloxegol in a population of 1247 healthy subjects and patients. Br J Clin Pharmacol 81(1):89-100 (2016).
Altier C & Zamponi G W. Opioid, cheating on its receptors, exacerbates pain. Nat Neurosci 9: 1465-1467 (2006).
Angst M & Clark JD. Opioid-induced Hyperalgesia: A Qualitative Systematic Review. Anesthesiology 104: 570-587 (2006).
Bardoni R. et al. Delta opioid receptors presynaptically regulate cutaneous mechanosensory neuron input to the spinal cord dorsal horn. Neuron 81: 1312-1327 (2014).
Blanchard R & Blanchard D. Passive and active reactions to fear-eliciting stimuli. J Comp Physiol Psychol 68: 129-135 (1969).
Bolles R. Species-specific defense reactions and avoidance learning. Psychol Rev 77: 32-48 (1970).
Bolles R & Fanselow M. A perceptual-defensive-recuperative model of fear and pain. Behav Brain Sci 3: 291-323 (1980).
Bovill J G, Sebel P S, Stanley TH. Opioid Analgesics in Anesthesia: With Special Reference to Their Use in Cardiovascular Anesthesia. Anesthesiology 61:731-755 (1984).
Caldeira, C. et al. Microglia change from a reactive to an age-like phenotype with the time in culture. Front. Cell. Neurosci. 8, 1-16 (2014).
Carroll I R, Angst Miss. & Clark J D. Management of perioperative pain in patients chronically consuming opioids. Reg. Anesth. Pain Med. 29, 576-591 (2004).
Cavanaugh D J et al. Restriction of transient receptor potential vanilloid-1 to the peptidergic subset of primary afferent neurons follows its developmental downregulation in nonpeptidergic neurons. J Neurosci 31: 10119-10127 (2011).
Chaplan S R, Bach F W, Pogrel J W, Chung J M & Yaksh T L. Quantitative assessment of tactile allodynia in the rat paw. J. Neurosci. Methods 53: 55-63 (1994).
Chen S R, Prunean A, Pan H M L, Welker K L & Pan H M L. Resistance to morphine analgesic tolerance in rats with deleted transient receptor potential vanilloid type 1-expressing sensory neurons. Neuroscience 145: 676-685 (2007).
Chen, L. H., Hedegaard, H. & Warner, M. Drug-poisoning Deaths Involving Opioid Analgesics: United States, 1999-2011. NCHS Data Brief 1-8 (2014).
Chu L F, Angst M S & Clark D. Opioid-induced hyperalgesia in humans: molecular mechanisms and clinical considerations. Clin J Pain 24: 479-496 (2008).
Christie M J. Cellular neuroadaptations to chronic opioids: tolerance, withdrawal and addiction. Br J Pharmacol 154: 384-396 (2008).
Collett B J. Opioid tolerance: the clinical perspective. Br J Anaesth 81: 58-68 (1998).
Corder G et al. Constitutive μ-opioid receptor activity leads to long-term endogenous analgesia and dependence. Science 341, 1394-1399 (2013).
Darwin C. The expression of the emotions in man and animals. (Albemarle, 1872).
Dhawan B N, Cesselin F, Raghubir R, Reisine T, Bradley P B, Portoghese P S, and Hamon M. International Union of Pharmacology. XII. Classification of Opioid Receptors. Pharmac Rev 48(4), 567-590 (1996).
Drdla, R., Gassner, M., Gingl, E. & Sandkuhler, J. Induction of synaptic long-term potentiation after opioid withdrawal. Science 325, 207-210 (2009).
Fanselow M S. The postshock activity burst. Anim. Learn. Behav. 10: 448-454 (1982).
Ferrini F, Trang T & Mattioli T. Morphine hyperalgesia gated through microglia-mediated disruption of neuronal Cl-homeostasis. Nat. Neurosci 16: 183-92 (2013).
Fletcher D & Martinez V. Opioid-induced hyperalgesia in patients after surgery: a systematic review and a meta-analysis. Br J Anaesth 112:991-1004 (2014).
Foo, L. C. et al. Development of a method for the purification and culture of rodent astrocytes. Neuron 71, 799-811 (2011).
Frank R G & Pollack H A. Addressing the Fentanyl Threat to Public Health. N Engl J Med 376, 605-607 (2017).
Fukagawa H, Koyama T, Kakuyama M & Fukuda K. Microglial activation involved in morphine tolerance is not mediated by toll-like receptor 4. J. Anesth. 27, 93-97 (2013).
Gomez I, Fujita W, Chandrakala M V & Devi L A. Disease-specific heteromerization of G protein-coupled receptors that target drugs of abuse. Prog Mol Biol Transl Sci 117:207-265 (2013).
Guan Z. et al. Injured sensory neuron-derived CSF1 induces microglial proliferation and DAP12-dependent pain. Nat. Neurosci. 19: 1-10 (2015).
He S. Q. et al. Tolerance develops to the antiallodynic effects of the peripherally acting opioid loperamide hydrochloride in nerve-injured rats. Pain 154: 2477-2486 (2013).
Heinke, B., Gingl, E. & Sandkühler, J. Multiple targets of μ-opioid receptor-mediated presynaptic inhibition at primary afferent Aδ- and C-fibers. J. Neurosci. 31, 1313-1322 (2011).
Heinl, C., Drdla-Schutting, R., Xanthos, D. N. & Sandkuhler, J. Distinct Mechanisms Underlying Pronociceptive Effects of Opioids. J. Neurosci. 31, 16748-16756 (2011).
Hollmann M W, Strumper D, Herroeder S & Durieux M E. Receptors, G Proteins, and Their Interactions. Anesthesiology 103:1066-1078 (2005).
Horvath, R. J., Romero-Sandoval, E. A. & Leo, J. a De. Inhibition of microglial P2X4 receptors attenuates morphine tolerance, Iba1, GFAP and ?? opioid receptor protein expression while enhancing perivascular microglial ED2. Pain 150, 401-413 (2010).
Hutchinson MR. et al. Evidence that opioids may have toll-like receptor 4 and MD-2 effects. Brain. Behav. Immun. 24, 83-95 (2010).
Jagla C, Martus P & Stein C. Peripheral opioid receptor blockade increases postoperative morphine demands-A randomized, double-blind, placebo-controlled trial. Pain 155: 2056-2062 (2014).
Joseph E K, Reichling D B & Levine J D. Shared mechanisms for opioid tolerance and a transition to chronic pain. J Neurosci 30, 4660-4666 (2010).
Julius D & Basbaum AI. Molecular mechanisms of nociception. Nature 413:203-210 (2001).
Kalso E, Edwards J E, Moore R A & McQuay H J. Opioids in chronic non-cancer pain: Systematic review of efficacy and safety. Pain 112, 372-380 (2004).
Kao S-C, Zhao X, Lee C-Y, Atianjoh F E, Gauda E B, Yaster M, Tao Y-X et al. Absence of L opioid receptor mRNA expression in astrocytes and microglia of rat spinal cord. Neuroreport 23: 378-384 (2012).
Kemp T, Spike R C, Watt C & Todd A J. The mu-opioid receptor (MOR1) is mainly restricted to neurons that do not contain GABA or glycine in the superficial dorsal horn of the rat spinal cord. Neuroscience 75: 1231-8 (1996).
Kim D, Langmead B & Salzberg S L. HISAT: a fast spliced aligner with low memory requirements. Nat. Methods 12: 357-360 (2015).
Kronschläger, M. T. et al. Gliogenic LTP spreads widely in nociceptive pathways. Science 354:1144-1148 (2016).
Madisen L et al. A toolbox of Cre-dependent optogenetic transgenic mice for light-induced activation and silencing. Nat. Neurosci. 15: 793-802 (2012).
Manglik, A. et al. Structure-based discovery of opioid analgesics with reduced side effects. Nature 537:185-190 (2016).
Mansour A, Fox C A, Akil H & Watson S. J. Opioid-receptor mRNA expression in the rat CNS: anatomical and functional implications. Trends Neurosci 18, 22-29 (1995).
Mao, J. Opioid-induced abnormal pain sensitivity: Implications in clinical opioid therapy. Pain 100, 213-217 (2002).
Martin W R. History and development of mixed opioid agonists, partial agonists and antagonists. Br J Clin Pharmac 7:273S-279S (1979).
Matthes H W, Maldonado R, Simonin F, Valverde O, Slowe S, Kitchen I, Befort K, Dierich A, Le Meur M, Dolle P, Tzavara E, Hanoune J, Roques B, Kieffer B L. Loss of morphine-induced analgesia, reward effect and withdrawal symptoms in mice lacking the mu-opioid-receptor gene. Nature 383, 819-823 (1996).
Mattioli, T. A. et al. Toll-like receptor 4 mutant and null mice retain morphine-induced tolerance, hyperalgesia, and physical dependence. PLoS One 9, 1-14 (2014).
Mogil J S. Animal models of pain: progress and challenges. Nat. Rev. Neurosci. 10: 283-294 (2009).
National Center for Biotechnology Information: 2004-2017. Available from https://www.ncbi.nlm.nih.gov/gene/4988
Pasternak G W & Pan Y-X. Mu Opioids and Their Receptors: Evolution of a Concept. Pharmac Reviews 65(4), 1257-1317 (2013).
Pasternak G W. Opioids and their receptors: Are we there yet? Neuropharmacology 76, 198-203 (2014).
Picelli S. et al. Smart-seq2 for sensitive full-length transcriptome profiling in single cells. Nat. Methods 10, 1096-8 (2013).
Pizzo A, Clark P M, Pokras N & Carter O. Institute of Medicine. Relieving Pain in America: A Blueprint for Transforming Prevention, Care, Education, and Research. Washington, D.C.: The National Academies Press, 2011.
Poelaert J. et al. Treatment with prolonged-release oxycodone/naloxone improves pain relief and opioid-induced constipation compared with prolonged-release oxycodone in patients with chronic severe pain and laxative-refractory constipation. Clin. Ther. 37: 784-792 (2015).
Portenoy R K et al. Subcutaneous methylnatrexone for the treatment of opioid-induced constipation in patients with advanced illness: a double-blind, randomized, parallel group, dose-ranging study. J Pain Symptom Manage 35:458-468 (2008).
Quinlan A. R. & Hall I. M. BEDTools: A flexible suite of utilities for comparing genomic features. Bioinformatics 26, 841-842 (2010).
Raghavendra V, Tanga F Y & DeLeo J A. Attenuation of Morphine Tolerance, Withdrawal Induced Hyperalgesia, and Associated Spinal Inflammatory Immune Responses by Propentofylline in Rats. Neuropsychopharmacology 29: 327-334 (2004).
Reichling D. B. & Levine J. D. Critical role of nociceptor plasticity in chronic pain. Trends Neurosci. 32, 611-618 (2009).
Rescola R. Pavlovian conditioned fear in Sidman avoidance learning. J Comp Physiol Psychol 65, 55-60 (1968).
Rosow C E et al. Reversal of opioid-induced bladder dysfunction by intravenous naloxone and methylnaltrexone. Clin. Pharmacol. Ther. 82: 48-53 (2007).
Rivat C & Ballantyne J. The dark side of opioids in pain management: basic science explains clinical observation. Pain Reports 1(2): e570 (2016).
Roeckel L-A, Le Coz G-M, Gaveriaux-Ruff C & Simonin F. Opioid-induced Hyperalgesia: Cellular and Molecular Mechanisms. Neuroscience 338: 160-182(2016).
Ruscheweyh, R., Wilder-Smith, O., Drdla, R., Liu, X.-G. & Sandkühler, J. Long-term potentiation in spinal nociceptive pathways as a novel target for pain therapy. Mol. Pain 7, 20 (2011).
Russell, J., Bass, P., Goldberg, L. I., Schuster, C. R. & Merz, H. Antagonism of gut, but not central effects of morphine with quaternary narcotic antagonists. Eur. J. Pharmacol. 78, 255-261 (1982).
Scherrer, G. et al. Dissociation of the opioid receptor mechanisms that control mechanical and heat pain. Cell 137, 1148-1159 (2009).
Simon L S. Relieving pain in America: a blueprint for transforming prevention, care, education, and research. J Pain Pall Care Pharmacother 26:197-198 (2012).
Solway, B., Bose, S. C., Corder, G., Donahue, R. R. & Taylor, B. K. Tonic inhibition of chronic pain by neuropeptide Y. Proc. Natl. Acad. Sci. U.S.A 108, 7224-7229 (2011).
Sorge, R. E. et al. Olfactory exposure to males, including men, causes stress and related analgesia in rodents. Nat. Methods 11, 629-632 (2014).
Stein C et al. Peripheral mechanisms of pain and analgesia. Brain Res. Rev. 60, 90-113 (2009).
Stiene-Martin, A., Zhou, R. & Hauser, K. F. Regional, developmental, and cell cycle-dependent differences in mu, delta, and kappa-opioid receptor expression among cultured mouse astrocytes. Glia 22, 249-59 (1998).
Terrando, N. et al. Stimulation of the α7 nicotinic acetylcholine receptor protects against neuroinflammation after tibia fracture and endotoxemia in mice. Mol. Med. 20, 667-75 (2015).
Thomas J. et al. Methylnaltrexone for opioid-induced constipation in advanced illness. N. Engl. J. Med. 358, 2332-2343 (2008).
Trang T, Al-Hasani R, Salvemini D, Salter M W, Gutstein H, and Cahill C M. Pain and Poppies: The Good, the Bad, and the Ugly of Opioid Analgesics. J Neurosci 35, 13879-13888 (2015).
Trapnell, C. et al. Transcript assembly and quantification by RNA-Seq reveals unannotated transcripts and isoform switching during cell differentiation. Nat. Biotechnol. 28, 511-515 (2010).
Turchan-Cholewo, J. et al. Cell-specific actions of HIV-Tat and morphine on opioid receptor expression in glia. J. Neurosci. Res. 86, 2100-10 (2008).
Usoskin, D. et al. Unbiased classification of sensory neuron types by large-scale single-cell RNA sequencing. Nat. Neurosci. 18, 145-153 (2014).
Varga E V, Yamamura H I, Rubenzik M K, Stropova D, Navratilova E, Roeske W R. Molecular mechanisms of excitatory signaling upon chronic opioid agonist treatment. Life Sci 74:299-311 (2003).
Volkow N & McLellan T. Opioid Abuse in Chronic Pain—Misconceptions and Mitigation Strategies. N Engl J Med 374:1253-1263 (2016).
Wang Y, Barker K, Shi S, Diaz M, Mo B, Gutstein H B. Blockade of PDGFR-13 activation eliminates morphine analgesic tolerance. Nat Med 18: 385-387 (2012).
Watkins L R, Hutchinson M R, Rice K C & Maier S F. The ‘Toll’ of Opioid-Induced Glial Activation: Improving the Clinical Efficacy of Opioids by Targeting Glia. Trends Pharmacol Sci 30: 581-591 (2009).
Webster L R. et al. Analysis of opioid-mediated analgesia in phase III studies of methylnaltrexone for opioid-induced constipation in patients with chronic noncancer pain. J. Pain Res. 8, 771-780 (2015).
Weibel, R. et al. Mu Opioid Receptors on Primary Afferent Nav1.8 Neurons Contribute to Opiate-Induced Analgesia: Insight from Conditional Knockout Mice. PLoS One 8, 1-18 (2013).
Williams J T, Ingram S L, Henderson G, Chavkin C, von Zastrow M, Schulz S, Koch T, Evans CJs, and Christie M J. Regulation of μ-opioid receptors: desensitization, phosphorylation, internalization, and tolerance. Pharm. Reviews 65: 223-254 (2013).
Woolf, C. J. Long term alterations in the excitability of the flexion reflex produced by peripheral tissue injury in the chronic decerebrate rat. Pain 18, 325-343 (1984).
Yu CS. et al. Safety and efficacy of methylnaltrexone in shortening the duration of postoperative ileus following segmental colectomy: Results of two randomized, placebo controlled phase 3 trials. Dis. Colon Rectum 54: 570-578 (2011).
Zacny J P, Wroblewski K & Coalson D W. Methylnaltrexone: its pharmacological effects alone and effects on morphine in healthy volunteers. Psychopharmacology (Berl). 232: 63-73 (2015).
Zhang, Y. et al. Essential role of toll-like receptor 2 in morphine-induced microglia activation in mice. Neurosci. Lett. 489, 43-47 (2011).
Zhang, Y. et al. An RNA-Sequencing Transcriptome and Splicing Database of Glia, Neurons, and Vascular Cells of the Cerebral Cortex. 34, 1-19 (2014).
Zhao, Y. L., Chen, S. R., Chen, H. & Pan, H. L. Chronic opioid potentiates presynaptic but impairs postsynaptic N-methyl-D-aspartic acid receptor activity in spinal cords: Implications for opioid hyperalgesia and tolerance. J. Biol. Chem. 287, 25073-25085 (2012).
Zhou, H.-Y. Y., Chen, S.-R. R., Chen, H. & Pan, H.-L. L. Opioid-induced long-term potentiation in the spinal cord is a presynaptic event. J Neurosci 30, 4460-4466 (2010).

What is claimed is:

1. A method of attenuating or preventing μ-opioid receptor mediated tolerance or opioid-induced hyperalgesia in a subject in need of acute or chronic opioid treatment for pain or in need of opioid anesthesia, comprising
ab initio administering to the subject a therapeutically effective amount of a composition including at least one peripherally acting μ-opioid antagonist and at least one μ-opioid agonist given concurrently or in essentially simultaneous sequence,
wherein the administering of the composition is effective to attenuate or prevent μ-opioid receptor mediated tolerance or opioid-induced hyperalgesia in said subject as evidenced by a decreased occurrence of symptoms generally associated with μ-opioid receptor mediated tolerance or hyperalgesia.

2. The method of claim 1, wherein the composition is provided in a delayed release formulation or a sustained release formulation.

3. The method of claim 2, wherein the delayed release formulation delays the peak concentration of the antagonist and the agonist in blood by 30 minutes to 12 hours from the time of administration.

4. The method of claim 2, wherein the sustained release formulation maintains a therapeutically effective dose of the antagonist and agonist for hours or days following administration.

5. The method of claim 1, wherein the composition is selected in contemplation of the respective pharmacokinetic properties of the antagonist and the agonist so that effective blood concentrations of the antagonist and agonist exist from treatment start on.

6. The method of claim 1, wherein the agonist is selected from the group consisting of morphine, fentanyl, fentanyl analog, methadone, buprenorphine, oxycodone, and hydromorphone, and wherein the antagonist is selected from the group consisting of methylnaltrexone (bromide) and naloxegol.

7. A pharmaceutical composition comprising at least one peripherally acting μ-opioid receptor antagonist and at least one μ-opioid receptor agonist for use in the preparation of a kit or medicament for attenuating or preventing μ-opioid receptor mediated tolerance or opioid-induced hyperalgesia in a subject in need of acute or chronic opioid treatment for pain OR in need of general anesthesia, wherein ab initio administration of a therapeutically effective amount of the kit or medicament to the subject attenuates or prevents symptoms generally associated with μ-opioid receptor mediated tolerance or opioid-induced hyperalgesia.

8. The composition of claim 7, comprising an agonist that is selected from the group consisting of morphine, fentanyl, fentanyl analog, methadone, buprenorphine, oxycodone, and hydromorphone, and comprising an antagonist that is selected from the group consisting of methylnaltrexone (bromide) and naloxegol.

9. A pharmaceutical composition comprising at least one peripherally acting μ-opioid receptor antagonist and at least one μ-opioid receptor agonist for use in the preparation of a kit or medicament for increasing a subject's pain threshold, wherein ab initio administration of a therapeutically effective amount of the kit or medicament to the subject reduces the amount of the μ-opioid receptor agonist needed to reduce pain perception during opioid anesthesia.

10. The composition of claim 9, comprising an agonist that is selected from the group consisting of morphine, fentanyl, fentanyl analog, methadone, buprenorphine, oxycodone, and hydromorphone, and comprising an antagonist that is selected from the group consisting of methylnaltrexone (bromide) and naloxegol.

11. A method of increasing pain threshold in a subject in need thereof, said method comprising administering the pharmaceutical composition of claim 9 to the subject wherein the administering of the composition is effective in reducing the amount of agonist needed to reduce pain perception during opioid anesthesia.


SEQUENCE LISTING (2 sequences)

SEQ ID NO: 1 - DNA, 24 bp, Artificial Sequence
  Primer
accagtacatggactggatgtgcc

SEQ ID NO: 2 - DNA, 24 bp, Artificial Sequence
  Primer
gagacaaggctctgaggatagtaa